(19) United States
(12) Patent Application Publication
(10) Pub. No.: US 2015/0368209 A1
(43) Pub. Date: Dec. 24, 2015

(54) 4-Pyrimidinylamino-benzenesulfonamide derivatives and their use for the inhibition of polo-like kinase 1 (PLK1) for the treatment of cancer and their use for the treatment of bacterial infections

(71) Applicants: VICHEM CHEMIE KUTATÓ KFT., Budapest (HU); ECOLE POLYTECHNIQUE FÉDÉRALE DE LAUSANNE (EPFL), Lausanne (CH)

(72) Inventors: Zoltán GREFF, Budapest (HU); Zoltán VARGA, Budapest (HU); György KÉRI, Budapest (HU); László ÕRFI, Budapest (HU); János PATÓ, Budapest (HU); Péter BÁNHEGYI, Budapest (HU); Csaba SZÁNTAI-KIS, Budapest (HU); Stewart COLE, Ecublens (CH); Ruben HARTKOORN, Ecublens (CH); Rita Edina SZÉKELY, Budapest (HU)

(73) Assignees: École Polytechnique Fédérale de Lausanne (EPFL), Lausanne (CH); Vichem Chemie Kutató Kft, Budapest (HU)

(21) Appl. No.: 14/759,247

(22) PCT Filed: Jan. 7, 2014
(86) PCT No.: PCT/HU2014/000002

§ 371 Date: Jul. 6, 2015

(30) Foreign Application Priority Data
Jan. 7, 2013 (HU) P1300007

(51) Int. Cl. C07D 239/42; C07D 413/14; C07D 417/12; C07D 413/12

(52) U.S. Cl. CPC C07D 239/42; C07D 413/12; C07D 413/14; C07D 417/12

(57) ABSTRACT

The present invention relates to 4-pyrimidinylamino-benzenesulfonamide derivatives of general formula (I) and pharmaceutically acceptable salts, solvates, hydrates, regioisomeric and polymorphic forms thereof, processes for manufacturing of them, the use of them, as well as pharmaceutical compositions containing at least one of them as pharmaceutically active agent(s) together with pharmaceutically acceptable carrier, excipient and/or diluents, especially for the inhibition of polo-like kinases (PLKs) and the treatment of cancer. Said 4-pyrimidinylamino-benzenesulfonamide compounds have been also identified as new drug candidates for the prevention and/or treatment of infectious diseases like bacterial diseases e.g. tuberculosis, including the currently multidrug-resistant tuberculosis (MDR-TB), extensively drug-resistant tuberculosis (XDR-TB) as well as for preventing tuberculosis.

### FIELD OF THE INVENTION

The present invention relates to 4-pyrimidinylamino-benzenesulfonamide derivatives of general formula (I) and pharmaceutically acceptable salts, solvates, hydrates, regioisomeric and polymorphic forms thereof, processes for manufacturing of them, the use of them, as well as pharmaceutical compositions containing at least one of them as pharmaceutically active agent(s) together with pharmaceutically acceptable carrier, excipient and/or diluents, especially for the inhibition of polo-like kinases (PLKs) and the treatment of cancer. Said 4-pyrimidinylamino-benzenesulfonamide compounds have been also identified as new drug candidates for the prevention and/or treatment of infectious diseases like bacterial diseases e.g. tuberculosis, including the currently multidrug-resistant tuberculosis (MDR-TB), extensively drug-resistant tuberculosis (XDR-TB) as well as for preventing tuberculosis.

### BACKGROUND OF THE INVENTION

Cancers are the major cause of death in humans. Many ways like surgery, radiation and chemotherapy are used to fight cancers. Antimitotic agents are one form of chemotherapy for solid tumors and hematologic malignancies. However current antimitotics (taxanes, vinca alkaloids) affect both dividing and non-dividing cells. Tumors can be characterized as subpopulations of cells which divide autonomously resulting the control of cell division—mitosis—partially or completely damaged. The consequence of the loss of cell cycle control is the excessive cell division activity and uncontrolled growth. Insufficient susceptibility to known medicines of many tumor types requires the development of novel compounds as chemotherapeutic agents interfering with cancer cell cycle and/or proliferation.

The subject of the present invention is novel PLK1 inhibitors relating to aminopyrimidin compounds. It is known that PLK1 (member of the polo like kinase family) the human orthologue of polo kinase of Drosophila is a key regulator kinase of mitosis and expressed only in dividing cells, mostly in M-phase. Although four different PLKs family members are described in humans, the inhibition of the enzymatic activity of PLK1 is sufficient to induce G2/M cell cycle block and apoptosis in tumor cell lines and tumor regression in xenograft models. In addition, for the other PLKs, a tumor suppressor function has been described and PLK2 and PLK3—but not PLK1—are reported to be expressed in non-proliferating, differentiated post mitotic cells, like neurons, indicating a possible better safety profile for a PLK1 specific compound. It is also proven that inhibiting the function of PLK1 with anti-sense oligonucleotides, small interfering RNAs (siRNA), or short hairpin RNA results in decreased tumor-derived cell survival and inhibited tumor growth in animal models. Overexpression of PLK1 has been described in many tumors types: breast cancer, colorectal cancer, esophagus and stomach cancer, endometrial carcinomas, head and neck squamous cell carcinomas, non-small cell lung cancer, ovarian cancer, pancreatic cancer and skin cancer among others.

It has now been found that the aminopyrimidine compounds described in detail below are characterized by surprising and advantageous properties such as, among others, the selective inhibition of PLK1 enzyme. It can be expected that among these PLK1 inhibitors there will be compounds that selectively inhibit proliferation and induce cell death in proliferating cancer cells while being inactive on arrested cells. Moreover it was observed that many of the pyridopyrimidinone compounds arrest proliferating cancer cells in mitosis.

### REFERENCES

Degenhardt Y, Lampkin T (2010). Targeting Polo-like Kinase in cancer therapy. Clin Cancer Res; 16(2):384-9.
Strebhardt K, et al., Nat Rev Cancer 2006; 6(4):321-30
WO 2009/112524 Pyridopyrimidines as plk1 (polo-like kinase) inhibitors

As it was mentioned above, the 4-pyrimidinylamino-benzenesulfonamide compounds according to the present invention also can be applied for the prevention and/or treatment of infectious diseases (especially bacterial diseases like e.g. mycobacterial diseases) like tuberculosis, including the currently multidrug-resistant tuberculosis (MDR-TB), extensively drug-resistant tuberculosis (XDR-TB) as well as for preventing tuberculosis.

Tuberculosis (TB) is a common and often deadly infectious disease caused by mycobacteria, usually Mycobacterium tuberculosis in humans. (Kumar et al. (2007) Robbins Basic Pathology (8th ed., Elsevier) pp. 516-522.) The M. tuberculosis complex includes four other TB-causing mycobacteria: Mycobacterium bovis, Mycobacterium africanum, Mycobacterium canetti and Mycobacterium microti. (Soolingen et al. (1997) Int. J. Syst. Bacteriol. 47 (4): 1236-45.) M. africanum is not widespread, but in parts of Africa it is a significant cause of TB. (Niemann et al. (2002) J. Clin. Microbiol. 40 (9): 3398-3405.; Niobe-Eyangoh et al. (2003) J. Clin. Microbiol. 41 (6): 2547-53.) M. bovis was once a common cause of TB, but the introduction of pasteurized milk has largely eliminated this as a public health problem in developed countries. (Thoen et al. (2006) Vet. MicrobioL 112 (2-4): 339-45.) M. canetti is rare and seems to be limited to Africa, although a few cases have been seen in African emigrants. (Pfyffer et al. (1998) Emerging Infect. Dis. 4 (4): 631-4.) M. microti is mostly seen in immunodeficient people, although it is possible that the prevalence of this pathogen has been underestimated. (Niemann et al. (2000) Emerg Infect Dis 6 (5): 539-42.)

Other known pathogenic mycobacteria include Mycobacterium leprae, Mycobacterium avium, Mycobacterium kansasii, Mycobacterium marinum, Mycobacterium scrofulaceum, Mycobacterium ulcerans Mycobacterium fortuitum, and Mycobacterium abscessus, and related species. All these mycobacteria, except M. leprae, are part of the nontuberculous mycobacteria (NTM) group. Nontuberculous mycobacteria cause neither TB nor leprosy, but they do cause pulmonary diseases resembling TB. The most common presentation of M. kansasii infection is a chronic pulmonary infection that resembles pulmonary tuberculosis. However, it may also infect other organs. M. kansasii infection is the second-most-common nontuberculous opportunistic mycobacterial infection associated with AIDS, surpassed only by M. avium complex (MAC) infection. For this reason, the incidence of M. kansasii infection has increased because of the HIV/AIDS epidemic. M. avium is a slow-growing bacterium found in the soil and in dust particles that causes tuberculosis in birds and swine and is responsible for the M. avium complex in humans. MAC is the most common cause of infection by nontuberculous mycobacteria in patients with AIDS (emedicine.medscape.com/article/222664-overview). M. marinum is a free-living bacterium, which causes opportunistic infections in humans. Is an atypical mycobacterium species found in cold or warm, fresh or salted water (Wolinsky, E. 1992. Mycobacterial diseases other than tuberculosis. Clin. Inf. Dis. 15:1-12.) M. marinum infection occurs following skin and soft-tissue injuries that are exposed to an aquatic environment or marine animals. The infection usually presents as a localized granuloma but can evolve into an ascending lymphangitis that resembles sporotrichosis or can spread to deeper tissues. M. scrofulaceum causes cervical lymphadenitis in children and very rarely pulmonary disease. (hopkins-abxguide.org) M. ulcerans is a very slow-growing mycobacterium derived from M. marinum, that classically infects the skin and subcutaneous tissues, giving rise to indolent nonulcerated (nodules, plaques) and ulcerated lesions (MacCallum, P., J. et al. (1948) “A new mycobacterial infection in man.” JPB LX: 93-122.) In many areas, M. ulcerans infection has only occurred after significant environmental disturbance. Because all major endemic foci are in wetlands of tropical or subtropical countries, environmental factors must play an essential role in the survival of the etiologic agent. M. fortuitum has a worldwide distribution and can be found in natural and processed water, sewage, and dirt. It is uncommon for it to cause lung disease. M. fortuitum can cause local cutaneous disease, osteomyelitis (inflammation of the bone), joint infections, and ocular disease after trauma. It is a rare cause of lymphadenitis (emedicine.medscape.com/article/222918-overview). The emerging pathogen, M. abscessus and its close relatives Mycobacterium massiliense and M. bolletti, is of growing concern. Infections with this group of bacteria are increasingly common in the immunodepressed population and are of considerable importance among cystic fibrosis patients as there are very few effective drugs available for treatment and the clinical outcome is poor (Olivier et al. (2003) Am J Respir Crit Care Med (167): 828-834). M. leprae, also known as Hansen's bacillus, is a bacterium that causes leprosy (Hansen's disease) (Ryan K J, Ray CG (editors) (2004) Sherris Medical Microbiology (4th ed.). McGraw Hill. pp. 451-3.).

A third of the world's population is thought to be infected with M. tuberculosis, and new infections occur at a rate of about one per second. (Jasmer et al. (2002) N. Engl. J. Med. 347 (23): 1860-1866.) The proportion of people who become sick with tuberculosis each year is stable or falling worldwide but, because of population growth, the absolute number of new cases is still increasing. (Tuberculosis. World Health Organization. (2007) Fact sheet No 104.) In 2007 there were an estimated 13.7 million chronic active cases, 9.3 million new cases, and 1.8 million deaths, mostly in developing countries. (World Health Organization (2009) Epidemiology. Global tuberculosis control: epidemiology, strategy, financing. pp. 6-33.) In addition, more people in the developed world are contracting tuberculosis because their immune systems are compromised by immunosuppressive drugs, substance abuse, or AIDS.

Tuberculosis usually attacks the lungs but can also affect other parts of the body. It is spread through the air, when people who have the disease cough, sneeze, or spit. Most infections in humans result in an asymptomatic, latent infection and about one in ten latent infections eventually progresses to active disease. (Konstantinos, A (2010) Testing for tuberculosis. Australian Prescriber, 33:12-18.) When the disease becomes active, 75% of the cases are pulmonary TB, that is, TB in the lungs. In the other 25% of active cases, the infection moves from the lungs, causing other kinds of TB, collectively denoted extrapulmonary tuberculosis. This occurs more commonly in immunosuppressed persons and young children. Extrapulmonary infection sites include the pleura in tuberculosis pleurisy, the central nervous system in meningitis, the lymphatic system in scrofula of the neck, the genitourinary system in urogenital tuberculosis, and bones and joints in Pott's disease of the spine. An especially serious form is disseminated TB, more commonly known as miliary tuberculosis. Extrapulmonary TB may co-exist with pulmonary TB as well. (Centers for Disease Control and Prevention (CDC), Division of Tuberculosis Elimination. Core Curriculum on Tuberculosis: What the Clinician Should Know. 4th edition (2000))

The first effective drugs for treatment of TB were Streptomycin, isolated from Streptomyces griseus strains in 1943, and the semi-synthetic Rifampicin (from Streptomyces mediterranei). (FIG. 1.)

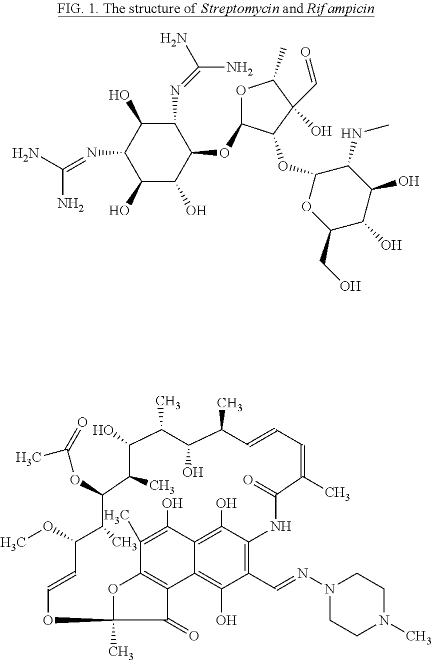

The current first-line TB drug regimen is more than 40 years old and consists primarily of isoniazid, ethambutol, pyrazinamide, and rifampicin. (FIG. 2.)

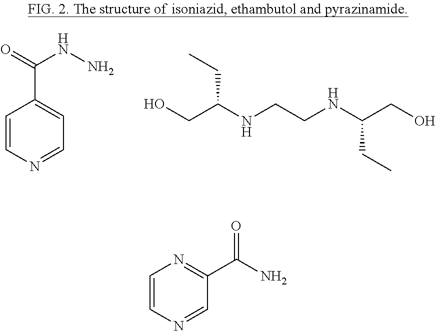

These antibiotics are effective in active, drug-susceptible TB, provided that patients complete the course. There is, however, poor patient compliance due to the cost of drugs, adverse effects, and especially to the long duration required for full treatment (6-12 months) and the required number of drug doses. Non-compliance has contributed to the appearance of multi-drug resistant (MDR) and extensively drug-resistant (XDR) TB strains. MDR-TB is resistant to isoniazid and rifampicin (at least), often taking a further two years to treat with second-line drugs (aminoglycosides, polypeptides, fluoroquinolones, thioamides, cycloserine, p-aminosalicylic acid) (Johnson, R. et al. (2006) Drug resistance in Mycobacterium tuberculosis. Curr. Issues Mol. Biol. 8, 97-112). XDR-TB also exhibits resistance to second-line drugs including fluoroquinolones and one of the following three drugs: capreomycin, kanamycin and amikacin, and is virtually incurable.

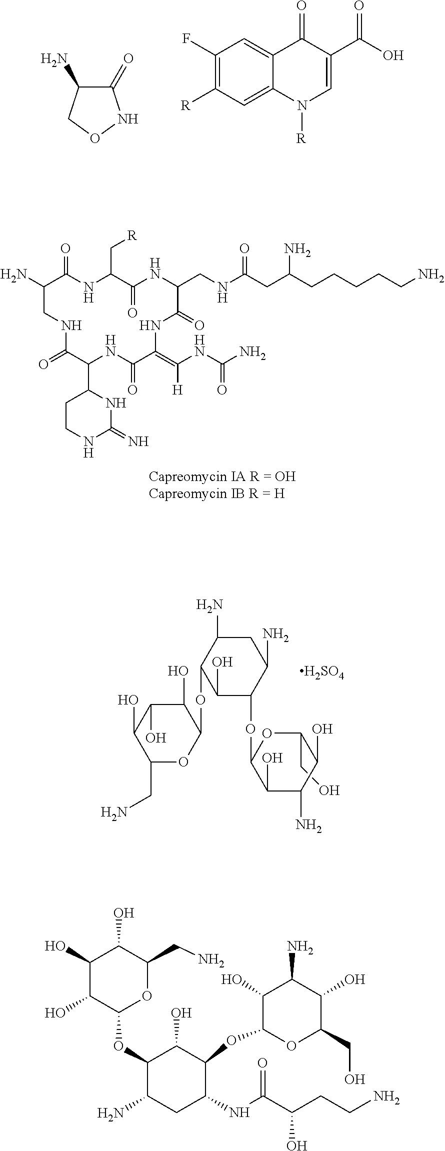

All the above reasons make a compelling case for the urgent need for new anti-TB drugs. In particular, shorter and more effective treatments would improve patient compliance and slow down the emergence of drug resistant strains.

Currently there are several anti-TB drug candidates in various phases of clinical development. (Table A.)

TABLE A
New promising anti-TB drug candidates. (www.clinicaltrials.gov)

| Structure | code / originator | effect | phase |
|---|---|---|---|
| 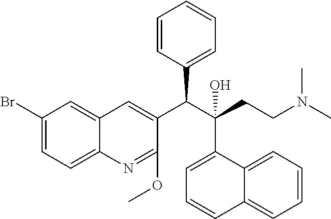 [SMILES: COc1nc2ccc(Br)cc2cc1[C@@H](c1ccccc1)[C@@](O)(CCN(C)C)c1cccc2ccccc12] | TMC207, Johnson&Johnson | inhibitor of bacterial ATP- synthetase | Phase II |
| 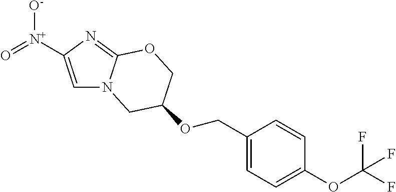 [SMILES: O=[N+]([O-])c1cn2c(n1)OC[C@@H](OCc1ccc(OC(F)(F)F)cc1)C2] | PA-824, Pathogenesis Global Alliance for TB Drug Development | inhibitor prodrug of mycolic acid and proteins | Phase II |
| 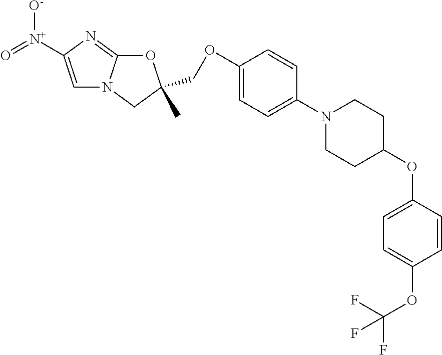 [SMILES: C[C@@]1(COc2ccc(N3CCC(Oc4ccc(OC(F)(F)F)cc4)CC3)cc2)Cn2cc([N+](=O)[O-])nc2O1] | OPC-67863, Otsuka Pharmaceutical | inhibitor prodrug of mycolic acid and proteins | Phase II |
| 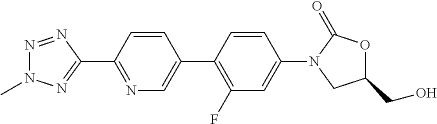 [SMILES: Cn1nnc(-c2ccc(-c3ccc(N4C[C@H](CO)OC4=O)cc3F)cn2)n1] | DA-7157, Dong- A Pharmaceutical | inhibitor of ribosomal protein synthesis | Phase IIa |
| 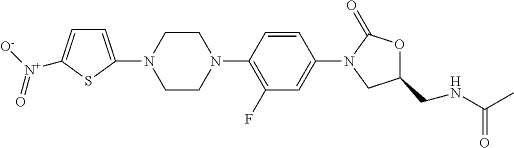 [SMILES: CC(=O)NC[C@H]1CN(c2ccc(N3CCN(c4ccc([N+](=O)[O-])s4)CC3)c(F)c2)C(=O)O1] | RBx8700 Ranbaxy Laboratories Limited | inhibitor of ribosomal protein synthesis | Preclinical phase |

### SUMMARY OF THE INVENTION

1. The present invention relates to compounds of the general formula (I) and pharmaceutically acceptable salts, solvates, hydrates, regioisomeric and polymorphic forms thereof:

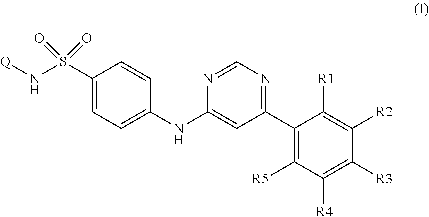

wherein

Q is a substituted or unsubstituted heterocyclyl having 5 or 12 ring member atoms where 1 to 3 of the ring member atoms are selected from the group of N, S and O and the other ring members are C, or alkanoyl, optionally substituted with one or more group selected from alkyl and oxo;

R1 to R5 are independently selected from the group of

a) hydrogen;

b) halogen;

c) optionally substituted alkyl, wherein the substituent is selected from the group of
optionally substituted aryloxy, wherein the substituent can be optionally halogen substituted alkyl or optionally halogen substituted alkoxy,
halogen,
aryl-alkoxy which is optionally substituted in the aryl part with optionally halogen substituted C₁₋₆ alkyl or optionally halogen substituted alkoxy;

d) optionally substituted alkoxy, wherein the substituent is selected from the group of
halogen,
optionally substituted aryl, wherein the substituent can be optionally halogen substituted alkyl or optionally halogen substituted alkoxy);

e) optionally substituted aryl;

e) aryloxy (preferably phenoxy);

f) nitrile;

g) amine, which optionally substituted with 1 or 2 alkyl or alkylcarbonyl (e.g.: acetamido);

h) carboxamide;

i) or any 2 adjacent groups of R1 to R5 form together an alkylenedioxy;

k) or any 2 adjacent groups of R1 to R5, together with the atom to which they are attached, form a condensed benzene ring.

2. A compound according to above point 1, wherein Q is selected from the following group:
3,4-dimethyl-isoxazol-5-yl,
5-methyl-[1,3,4]thiadiazol-2-yl,
5-methyl-isoxazol-3-yl,
3,4-dimethyl-isoxazol-5-yl.

3. A compound according to above point 1, wherein in the meaning of R1 to R5, in point c) the aryl-alkoxy is a benzyloxyalkyl group, e.g. benzyloxymethyl.

4. A compound according to above point 1, wherein in the meaning of R1 to R5, in point d) the alkoxy optionally substituted with aryl is a benzyloxy group.

5. 4. A compound according to above point 1, wherein in the meaning of R1 to R5, in point e) the aryloxy is a phenoxy group.

6. A compound according to any of above points 1 to 5 for use in the prevention and/or the treatment of cancer diseases.

7. A compound according to any of above points 1 to 5 for use in the prevention and/or the treatment of bacterial diseases, e.g. mycobacterial diseases.

8. A compound according for use according to above point 7 where the bacterial disease is tuberculosis.

9. Pharmaceutical composition containing as active ingredient one or more compound(s) of general formula (I) according to any of above points 1 to 5 together with one or more usual pharmaceutical auxiliary material(s).

10. Method for the prevention and/or the treatment of a cancerous disease where a compound of general formula (I) according to any of above points 1 to 5 is administered to an individual in need thereof.

11. Method for the prevention and/or the treatment of a bacterial diseases, especially mycobacterial diseases, e.g. tuberculosis, where a compound of general formula (I) according to any of above points 1 to 5 is administered to an individual in need thereof.

### DETAILED DESCRIPTION OF THE INVENTION

In the context of this description the phrase “cancer” embraces adenocarcinomas (breast, colon, colorectal and colorectal adenocarcinoma, epidermoid, lung bronchioalveolar and lung adenocarcinoma), the cancerous disease of the genital system (including uterine cervix, uterine corpus, ovary, vulva, vagina and other genital female, prostate, testis, penis and other genital male), digestive system (including esophagus, stomach, small intestine, colon, rectum, anus anal canal and anorectum, liver and intrahepatic bile duct, gallbladder and other biliary, pancreas, other digestive organs), respiratory system (including larynx, lung and bronchus, other respiratory organs), breast, urinary system (including urinary bladder, kidney and renal pelvis, ureter and other urinary organs), skin (excluding basal and squamous; including skin melanoma, other nonepithelial skin), endocrine system (including thyroid, other endocrine), oral cavity and pharynx (including tongue, mouth, pharynx, other oral cavity), brain and other nervous system, myeloma, soft tissue (including heart), bones and joints, eye and orbit, and the following diseases: lymphoma (including Hodgkin lymphoma, Non-Hodgkin lymphoma), leukemia (including acute lymphocytic leukemia, chronic lymphocytic leukemia, acute myeloid leukemia, chronic myeloid leukemia, other leukemia), especially acute T-cell leukemia, breast, colon, colorectal and colorectal adenocarcinoma, epidermoid, lung bronchioalveolar and lung adenocarcinoma, prostate.

In the context of this description the phrase “bacterial disease” (equal to “bacterial related disease”) embraces diseases caused by e.g. the following bacteria: Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Yersinia pestis);

also including Gram-positive bacteria, e.g. Staphylococcus aureus, Streptococcus pyogenes, Streptococcus pneumoniae, Corynebacterium and Listeria);

also including pathogenic genuses of Actinobacteria; e.g. genus Mycobacterium, including the species M. tuberculosis which causes tuberculosis and M. leprae which causes leprosy;

Corynebacterium, includes C. diphtheriae causing diphtheria; Nocardia which has several pathogenic species commonly causing nocardiosis);

also including Mycobacteria, e.g. M. tuberculosis (and its complex: MBTC), M. avium (and its complex: MAC), M. gordonae, M. avium paratuberculosis (which has been implicated in Crohn's disease), M. bovis, M. africanum, M. canetti, M. leprae (which causes leprosy), M. marinum, M. scrofulaceum, M. ulcerans (which causes the “Buruli”, or “Bairnsdale, ulcer”), M. microti, M. fortuitum-chelonei compex, M. branderi, M. cookii, M. celatum, M. bohemicum, M. haemophilum, M. malmoense, M. szulgai, M. lepraemurium, M. lepromatosis (another cause of leprosy), M. botniense, M. chimaera, M. conspicuum, M. doricum, M. farcinogenes, M. heckeshornense, M. intracellulare, M. lacus, M. monacense, M. montefiorense, M. murale, M. nebraskense, M. saskatchewanense, M. scrofulaceum, M. shimoidei, M. tusciae, M. xenopi, M. yongonense, M. intermedium, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diemhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense, M. tokaiense.

In the context of this description the phrase “mycobacterial disease” (equal to “mycobacterial related disease)” embraces Tuberculosis (TB) caused by mycobacteria, usually Mycobacterium tuberculosis in humans, including multi-drug resistant (MDR) and extensively drug-resistant (XDR) TB strains; leprosy caused by Mycobacterium leprae, and diseases related by one or more from the followings; Mycobacterium tuberculosis complex (MTBC) includes these four TB-causing mycobacteria: Mycobacterium bovis, Mycobacterium africanum, Mycobacterium canetti, Mycobacterium microti. Other known pathogenic mycobacteria include: Mycobacterium leprae, Mycobacterium avium, Mycobacterium kansasii, Mycobacterium massiliense, Mycobacterium bolletti, Mycobacterium marinum, Mycobacterium scrofulaceum, Mycobacterium ulcerans, Mycobacterium fortuitum, Mycobacterium caprae, Mycobacterium mungi, Mycobacterium orygis, Mycobacterium pinnipedii, Mycobacterium abscessus, and related species.

As used herein the term “heterocyclyl” means a group derived from a saturated, partially unsaturated or aromatic ring system having 5 to 12 ring member atoms where 1 to 3 of the ring member atoms are selected from the group of N, S and O and the other ring members are C [where N is nitrogen, 0 is oxygen, S is sulfur and C is carbon atom]. Preferably the heterocycle group has 5 or 6 (e.g. 5) ring member atoms where 1 to 3 of the ring member atoms (e.g. 1 or 2) is/are selected from the group of N, S and O and the other ring members are C, N and S are especially preferred, but here we underline that 0 is very close analogue of S from chemical point of view (they are in the same row of the Periodic Table of Elements). The heterocycle can be for example indolyl, indazolyl, 1,3-benzodioxolyl, furanyl, pyrrolyl, pyridinyl, quinolinyl, isoquinolinyl, pyranyl, oxazinyl, isoxalolyl, thiazinyl, thiadiazolyl, thienyl, imidazolyl, benzoimidazolyl, pyrazolyl, purinyl, where indolyl, indazolyl, isoxalolyl, 1,3-benzodioxolyl, pyridinyl, quinolinyl, thiadiazolyl, isoquinolinyl are preferred, especially isoxalolyl and thiadiazolyl. The following groups are especially preferred: 3,4-dimethyl-isoxazol-5-yl, 5-methyl-[1,3,4]thiadiazol-2-yl, 5-methyl-isoxazol-3-yl, 3,4-dimethyl-isoxazol-5-yl.

Those substituted heterocyclyl groups are also within the scope which contain one or more substituent(s) usually applied in the organic chemistry for substitution of heterocyclyl groups. So, the substituted heterocyclyl groups carry one or more, e.g. 1 to 4, or. 1 to 3 or 1 or 2 substituent(s), independently selected from e.g. the group of halogen, alkyl, hydroxyl, hydroxyalkyl, carboxyl, alkoxy, haloalkyl, nitro, sulphate, amino, acylamino, carboxylate, amide monoalkylamino, dialkylamino, alkylthio, alkylsulfinyl, alkylsulfonyl and cyano. The saturated, partially unsaturated or aromatic ring systems may contain 4 to 6 carbon atoms and 1 to 3 nitrogen atom(s), see e.g. morpholinyl, piperidinyl, piperazinyl, methylpiperazinyl [preferably the substituent is halogen, more preferably a saturated ring system contains 4 to 6 carbon atoms and 1 to 3 nitrogen and atom(s)], and the substituent may be selected from the group of carboxyl, alkoxy, haloalkyl, nitro, sulphate, amino, acylamino, carboxylate, amide monoalkylamino, dialkylamino, alkylthio, alkylsulfinyl, alkylsulfonyl and cyano, where C1-3 alkyl, e.g. methyl, halogen (e.g. fluoro) or a saturated ring system containing 4 to 6 carbon and 1 to 3 N (e.g. piperazinyl) are preferred. The alkyl substituent is especially preferred.

As used herein the term “aryl”, alone or in combinations means an aromatic monocyclic or multicyclic ring system comprising 6 to 14 carbon atoms, preferably 6 to about 10 carbon atoms, more preferably 6 carbon atoms, e.g. phenyl or naphthyl, especially phenyl.

Those substituted aryl groups are also within the scope which contain one or more substituent(s) [e.g. 1 to 5, or 1 to 4, or 1 to 3 or 1 or 2 substituent(s), independently selected from each other] usually applied in the organic chemistry for substitution of aryl groups. So, the substituted aryl groups carry one or more, preferably one to three substituent(s), independently selected from the group of halogen, optionally substituted alkyl (more preferably methyl and trifluoromethyl), optionally substituted alkoxy (more preferably methoxy), hydroxyl, carboxyl, carboxylate, haloalkyl, nitro, sulphate, amino, amide, acylamino, monoalkylamino, dialkylamino, alkylthio, alkylsulfinyl, alkylsulfonyl and cyano. The saturated, partially unsaturated or aromatic ring systems may contain 4 to 6 carbon atoms and 1 to 3 nitrogen atom(s) (see e.g. morpholinyl, piperazinyl, piperidinyl, methylpiperazinyl, piperidinyl;), and the substituent may be selected from the group of carboxyl, carboxylate, alkoxy, haloalkyl, nitro, sulphate, amino, amide, acylamino, monoalkylamino, dialkylamino, alkylthio, alkylsulfinyl, alkylsulfonyl and cyano, where alkyl (more preferably methyl and trifluoromethyl), halogen, hydroxyl, alkoxy (more preferably methoxy, optionally substituted with halogen, e.g. fluoro), nitro, carboxyl, carboxylate (more preferably methyl carboxylate), amino, amide, especially halogen, alkyl and alkoxy, e.g. alkyl and alkoxy optionally substituted with halogen.

As used herein, the term “aryloxy” means an aryl-O— group in which the aryl group is as previously described. Preferred example of the aryloxy groups is the phenoxy.

As used herein, the term “halogen” means fluorine, chlorine, bromine or iodine.

As used herein, the term “alkyl” alone or in combinations means a straight or branched-chain alkyl group containing from 1 to 6, preferably 1 to 5 carbon atom(s) (i.e. “C₁₋₆” or “C₁₋₅” alkyl groups), such as methyl, ethyl, propyl, isopropyl, butyl, sec-butyl, t-butyl and pentyl. In special cases this phrase can relate to alkyl groups containing from 1 to 4, or 1 to 3 or 1 or 2 carbon atom(s) (i.e. “C₁₋₄” or “C₁₋₃” or “C₁₋₂” alkyl groups).

Those substituted alkyl groups are also within the scope which contain one or more substituent(s) [e.g. 1 to 4, or. 1 to 3 or 1 or 2 substituent(s), independently selected from each others] usually applied in the organic chemistry for substitution of alkyl groups. So, the substituted alkyl groups carry one or more, preferably one or two substituent(s), independently selected from the group of halogen (resulting in e.g. trifloromethyl), aryl, aryloxy, hydroxyl, carboxyl, benzyloxy, alkoxy, nitro, sulphate, amino, acylamino, monoalkylamino, dialkylamino, alkylthio, alkylsulfinyl, alkylsulfonyl and cyano (nitrile), e.g. halogen and hydroxyl, especially halogen.

As used herein, the term “alkoxy” means an alkyl-O— group in which the alkyl group is as previously described. Non-limiting examples of suitable alkoxy groups include methoxy, ethoxy, n-propoxy, isopropoxy and n-butoxy, or halogenated derivatives thereof, e.g. trifluormethoxy. The bond to the parent moiety is through the ether oxygen. If the alkoxy group is substituted with halogen then it is named as haloalkoxy group.

As used herein, the term “carboxamide” means —C(O)NH₂ group.

As used herein, the term “alkylcarbonyl” or “alkanoyl” means a —C(O)—R group where R is an C₁₋₅ alkyl group. For example, an amino group can be substituted with such a group, resulting in e.g. an acetamido group.

As used herein, the term “alkylenedioxy” means a —O—(CH₂)ₙ—O— group, where n is 1, 2, 3 or 4, i.e. an “C₁₋₄ alkylenedioxy group”, where n=3 or 4 is preferred. When e.g. a phenyl is substituted with such a group, a saturated ring is condensed on it by this substituent.

The term “salt” means any ionic compound formed between one of the embodiments of the present invention and an acidic or basic molecule that can donate or accept ionic particle to/from its partner. The quaternary amine salts are also included.

Pharmaceutically acceptable (i.e., non-toxic, physiologically acceptable) salts are preferred, although other salts are also useful. Salts of the compounds of the formula (I) may be formed, for example, by reacting a compound of formula (I) with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hem isulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. Additionally, acids, which are generally considered suitable for the formation of pharmaceutically useful salts from basic pharmaceutical compounds are known.

The term “solvate” means a compound formed by the combination of solvent molecules with molecules or ions of the solute (solvation). Solute can be any of the embodiments of the present invention and the solvent can be water (forming hydrates) or any organic solvent.

Materials and Methods:

General Method

### Step A: Preparation of 4-(6-chloro-pyrimidin-4-ylamino)-N-(3,4-dimethyl-isoxazol-5-yl)-benzenesulfonamide

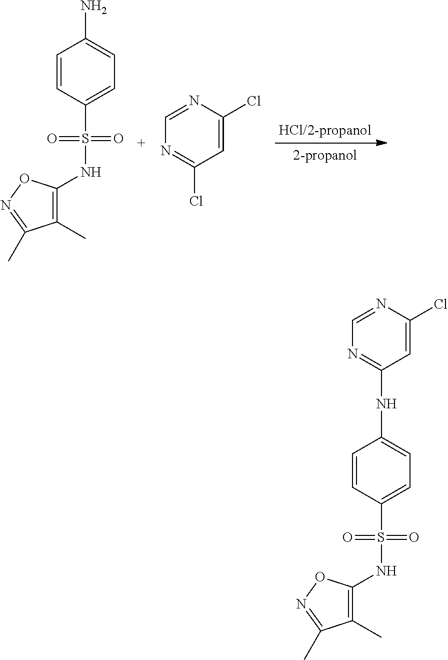 [SMILES: Cc1noc(NS(=O)(=O)c2ccc(N)cc2)c1C ; Clc1cc(Cl)ncn1 ; Cc1noc(NS(=O)(=O)c2ccc(Nc3cc(Cl)ncn3)cc2)c1C]

5.34 g (20.00 mmol) 4-amino-N-(3,4-dimethyl-isoxazol-5-yl)-benzenesulfonamide (Sulfisoxazole, available from Matrix Scientific, catalog nr.: 063874), 3.28 g (22.00 mmol) 4,6-dichloro-pyrimidine, and 15 ml 2-propanol saturated with HCl in 100 ml 2-propanol was refluxed for one hour. After cooling the reaction mixture to room temperature the solvent was evaporated, and the residue was treated with 75 ml water. The pH was changed to 6 using solid sodium-hydrogen-carbonate, and the solution was extracted five times with 75 ml ethyl acetate. The collected organic phase was washed with 50 ml brine, dried over magnesium-sulphate, and the solvent was evaporated. The crude product was refluxed for a half an hour in 75 ml acetonitrile, and after cooling to 0° C. the pure product was filtered off.

Yield: 4.90 g (65%)

### Step B: Procedure for the Suzuki Coupling Reaction

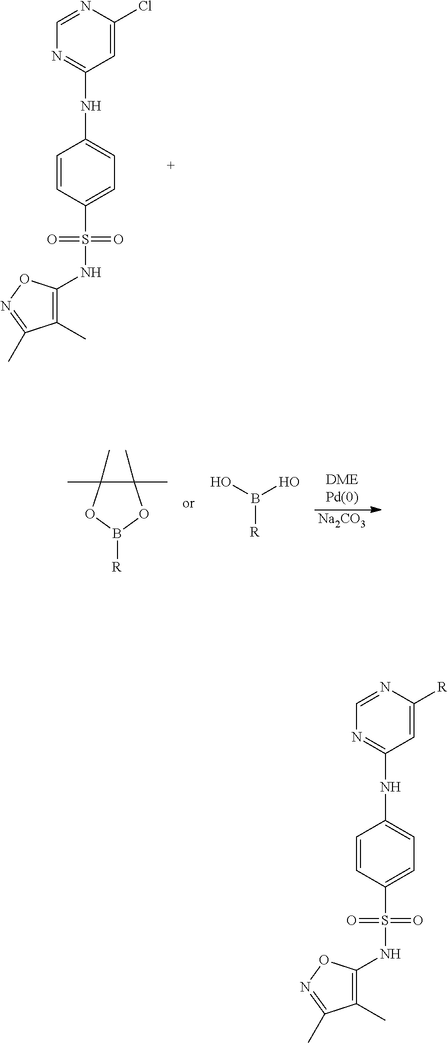

0.38 g (1.00 mmol) 4-(6-chloro-pyrimidin-4-ylamino)-N-(3,4-dimethyl-isoxazol-5-yl)-benzenesulfonamide and 0.06 g (0.05 mmol) tetrakis(triphenyl-phosphin)-palladium(0) in 50 ml 1,2-dimethoxyethane was stirred at room temperature for 1.5 hours under argon atmosphere. Then 1.10 mmol R-boronic acid or R-boronic acid ester, 0.21 g (2.00 mmol) sodium carbonate and 1.00 ml water were added into the reaction mixture, and it was refluxed under argon atmosphere for 2 to 24 hours. The reaction mixture was cooled to room temperature, quenched with 50 ml 1 M sodium-dihydrogen-phosphate buffer solution, and it was extracted three times with 50 ml ethyl-acetate. The organic phase was washed with 30 ml brine, treated with activated charcoal and magnesium-sulphate, was stirred for ten minutes, and after filtration was evaporated. The residue was crystallized from acetonitrile to give the product.

Analytical Characterization

All of the prepared compounds were characterized by the following analytical methods.

NMR

The 300 MHz ¹H-NMR analysis was performed with an apparatus of type Brucker AVANCE-300 at 25° C., exact frequency was 300.14 MHz. Generally DMSO-d₆ was used as solvent, exceptions given.

The 600 MHz ¹H-NMR and ¹³C-NMR spectra were recorded on a Varian Inova-600 MHz device at 25° C., the solvent was DMSO-d₆ (δ_{C}=39.50 and δ_{H}=2.50).

LCMS

The LCMS analysis was performed with a liquid chromatography mass-spectrometer Waters chromatograph with the following parameters:

Waters HPLC/MS:

MS detector: Method “A”: MicroMass ZMD
Method “B”: Waters SQD
UV detector: Waters 996 DAD
Separation module: Waters Alliance 2795

HPLC:
Column:
Waters XBridge C18, 50 mm×4.6 mm, 3.5 μm
Solvent I: Water/0.1% HCOOH
Solvent II: AcCN
Acetonitrile: Riedel-deHaën; G Chromasolv (34998)
Water: Mili-Q Academic
Formic acid: Riedel-deHaen; extra pure (27001)
Flow rate: 2 ml/min
Injection: 5 μg

Gradient:

| Time | Solv. I. | Solv. II. |
|---|---|---|
| 0.00 min | 95% | 5% |
| 0.50 min | 95% | 5% |
| 5.50 min | 5% | 95% |
| 6.00 min | 5% | 95% |
| 6.50 min | 95% | 5% |
| 7.00 min | 95% | 5% |

MS:
Ionization: ES⁺/ES⁻
Source block temperature: 110° C.
Desolvation temperature: 250° C.
Desolvation gas: 500 L/h
Cone gas: 80 L/h
Capillary voltage: 3000 V
Cone voltage: 30 V
Extractor voltage: 6 V
Rf lens voltage: 0.1 V
Scan: 80 to 1000 m/z in 1 sec.
Inter-scan delay: 0.1 s

TABLE 1
Identification of the prepared compounds.

| Example | Structure | Name | Formula |
|---|---|---|---|
| 1 | 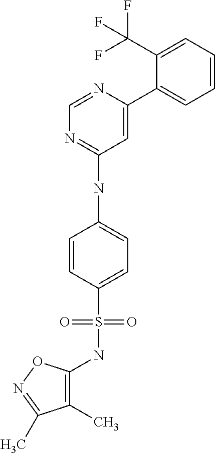 [SMILES: Cc1noc(NS(=O)(=O)c2ccc(Nc3cc(-c4ccccc4C(F)(F)F)ncn3)cc2)c1C] | N-(3,4-Dimethyl-isoxazol-5-yl)-4-[6-(2- trifluoromethyl-phenyl)-pyrimidin-4- ylamino]-benzenesulfonamide | C22H18F3N5O3S |
| 2 | 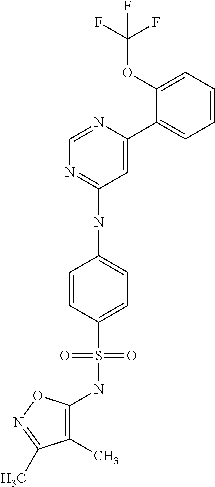 [SMILES: Cc1noc(NS(=O)(=O)c2ccc(Nc3cc(-c4ccccc4OC(F)(F)F)ncn3)cc2)c1C] | N-(3,4-Dimethyl-isoxazol-5-yl)-4-[6-(2- trifluoromethoxy-phenyl)-pyrimidin-4- ylamino]-benzenesulfonamide | C22H18F3N5O4S |
| 3 | 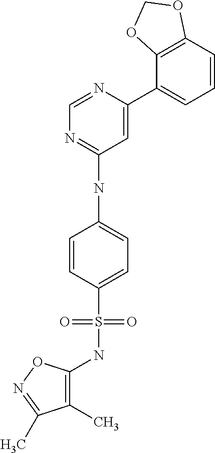 [SMILES: Cc1noc(NS(=O)(=O)c2ccc(Nc3cc(-c4cccc5c4OCO5)ncn3)cc2)c1C] | 4-(6-Benzo[1,3]dioxol-4-yl-pyrimidin-4- ylamino)-N-(3,4-dimethyl-isoxazol-5-yl)- benzenesulfonamide | C22H19N5O5S |
| 4 | 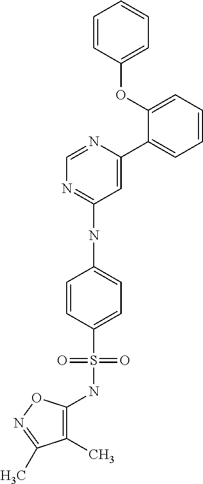 [SMILES: Cc1noc(NS(=O)(=O)c2ccc(Nc3cc(-c4ccccc4Oc4ccccc4)ncn3)cc2)c1C] | N-(3,4-Dimethyl-isoxazol-5-yl)-4-[6-(2- phenoxy-phenyl)-pyrimidin-4-ylamino]- benzenesulfonamide | C27H23N5O4S |
| 5 | 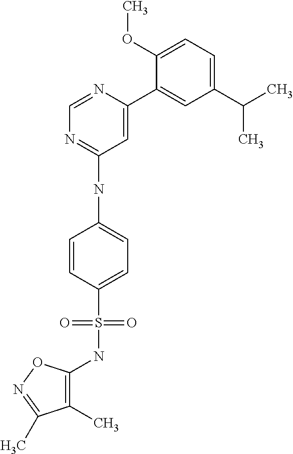 [SMILES: COc1ccc(C(C)C)cc1-c1cc(Nc2ccc(S(=O)(=O)Nc3onc(C)c3C)cc2)ncn1] | N-(3,4-Dimethyl-isoxazol-5-yl)-4-[6-(5- isopropyl-2-methoxy-phenyl)-pyrimidin-4- ylamino]-benzenesulfonamide | C25H27N5O4S |
| 6 | 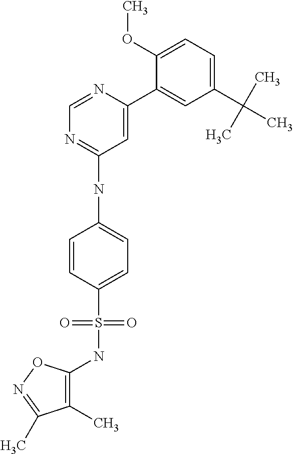 [SMILES: COc1ccc(C(C)(C)C)cc1-c1cc(Nc2ccc(S(=O)(=O)Nc3onc(C)c3C)cc2)ncn1] | 4-[6-(5-tert-Butyl-2-methoxy-phenyl)- pyrimidin-4-ylamino]-N-(3,4-dimethyl- isoxazol-5-yl)-benzenesulfonamide | C26H29N5O4S |
| 7 | 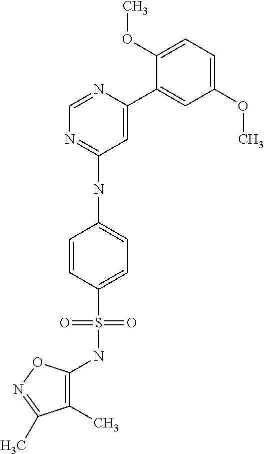 [SMILES: COc1ccc(OC)c(-c2cc(Nc3ccc(S(=O)(=O)Nc4onc(C)c4C)cc3)ncn2)c1] | 4-[6-(2,5-Dimethoxy-phenyl)-pyrimidin-4- ylamino]-N-(3,4-dimethyl-isoxazol-5-yl)- benzenesulfonamide | C23H23N5O5S |
| 8 | 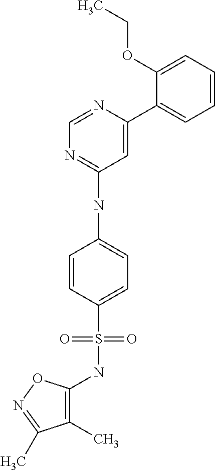 [SMILES: CCOc1ccccc1-c1cc(Nc2ccc(S(=O)(=O)Nc3onc(C)c3C)cc2)ncn1] | N-(3,4-Dimethyl-isoxazol-5-yl)-4-[6-(2- ethoxy-phenyl)-pyrimidin-4-ylamino]- benzenesulfonamide | C23H23N5O4S |
| 9 | 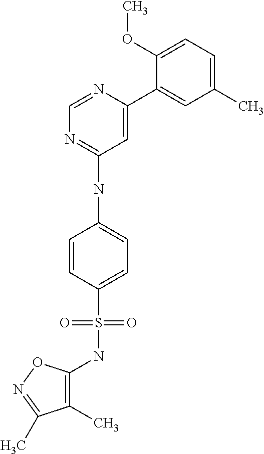 [SMILES: COc1ccc(C)cc1-c1cc(Nc2ccc(S(=O)(=O)Nc3onc(C)c3C)cc2)ncn1] | N-(3,4-Dimethyl-isoxazol-5-yl)-4-[6-(2- methoxy-5-methyl-phenyl)-pyrimidin-4- ylamino]-benzenesulfonamide | C23H23N5O4S |
| 10 | 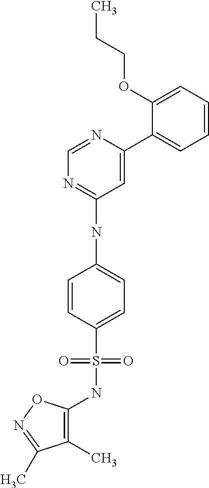 [SMILES: CCCOc1ccccc1-c1cc(Nc2ccc(S(=O)(=O)Nc3onc(C)c3C)cc2)ncn1] | N-(3,4-Dimethyl-isoxazol-5-yl)-4-[6-(2- propoxy-phenyl)-pyrimidin-4-ylamino]- benzenesulfonamide | C24H25N5O4S |
| 11 | 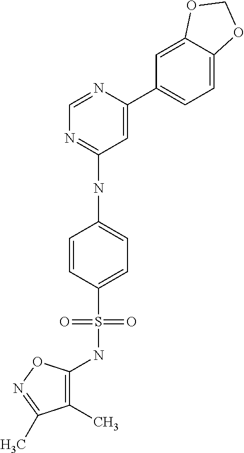 [SMILES: Cc1noc(NS(=O)(=O)c2ccc(Nc3cc(-c4ccc5c(c4)OCO5)ncn3)cc2)c1C] | 4-(6-Benzo[1,3]dioxol-5-yl-pyrimidin-4- ylamino)-N-(3,4-dimethyl-isoxazol-5-yl)- benzenesulfonamide | C22H19N5O5S |
| 12 | 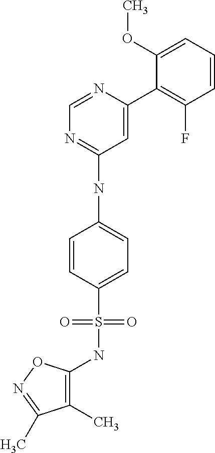 [SMILES: COc1cccc(F)c1-c1cc(Nc2ccc(S(=O)(=O)Nc3onc(C)c3C)cc2)ncn1] | N-(3,4-Dimethyl-isoxazol-5-yl)-4-[6-(2- fluoro-6-methoxy-phenyl)-pyrimidin-4- ylamino]-benzenesulfonamide | C22H20FN5O4S |
| 13 | 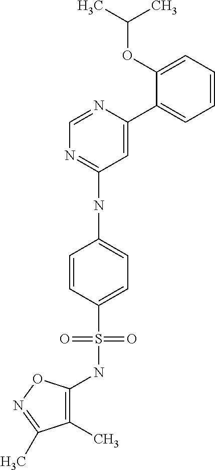 [SMILES: Cc1noc(NS(=O)(=O)c2ccc(Nc3cc(-c4ccccc4OC(C)C)ncn3)cc2)c1C] | N-(3,4-Dimethyl-isoxazol-5-yl)-4-[6-(2- isopropoxy-phenyl)-pyrimidin-4-ylamino]- benzenesulfonamide | C24H25N5O4S |
| 14 | 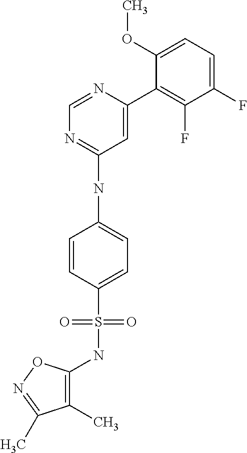 [SMILES: COc1ccc(F)c(F)c1-c1cc(Nc2ccc(S(=O)(=O)Nc3onc(C)c3C)cc2)ncn1] | 4-[6-(2,3-Difluoro-6-methoxy-phenyl)- pyrimidin-4-ylamino]-N-(3,4-dimethyl- isoxazol-5-yl)-benzenesulfonamide | C22H19F2N5O4S |
| 15 | 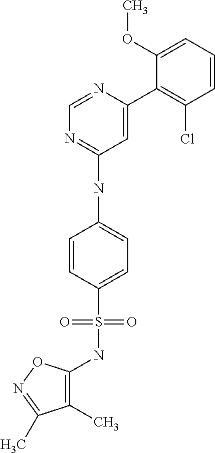 [SMILES: COc1cccc(Cl)c1-c1cc(Nc2ccc(S(=O)(=O)Nc3onc(C)c3C)cc2)ncn1] | 4-[6-(2-Chloro-6-methoxy-phenyl)- pyrimidin-4-ylamino]-N-(3,4-dimethyl- isoxazol-5-yl)-benzenesulfonamide | C22H20ClN5O4S |
| 16 | 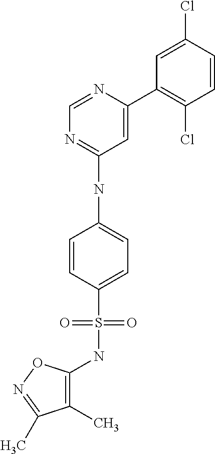 [SMILES: Cc1noc(NS(=O)(=O)c2ccc(Nc3cc(-c4cc(Cl)ccc4Cl)ncn3)cc2)c1C] | 4-[6-(2,5-Dichloro-phenyl)-pyrimidin-4- ylamino]-N-(3,4-dimethyl-isoxazol-5-yl)- benzenesulfonamide | C21H17Cl2N5O3S |
| 17 | 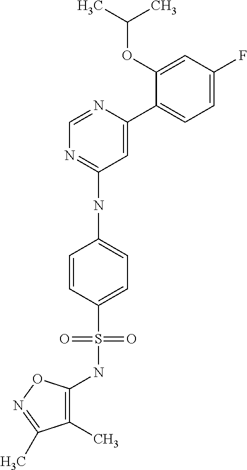 [SMILES: Cc1noc(NS(=O)(=O)c2ccc(Nc3cc(-c4ccc(F)cc4OC(C)C)ncn3)cc2)c1C] | N-(3,4-Dimethyl-isoxazol-5-yl)-4-[6-(4- fluoro-2-isopropoxy-phenyl)-pyrimidin-4- ylamino]-benzenesulfonamide | C24H24FN5O4S |
| 18 | 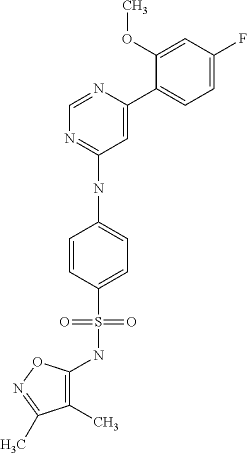 [SMILES: COc1cc(F)ccc1-c1cc(Nc2ccc(S(=O)(=O)Nc3onc(C)c3C)cc2)ncn1] | N-(3,4-Dimethyl-isoxazol-5-yl)-4-[6-(4- fluoro-2-methoxy-phenyl)-pyrimidin-4- ylamino]-benzenesulfonamide | C22H20FN5O4S |
| 19 | 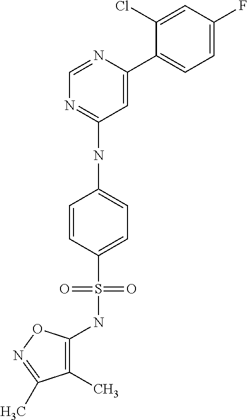 [SMILES: Cc1noc(NS(=O)(=O)c2ccc(Nc3cc(-c4ccc(F)cc4Cl)ncn3)cc2)c1C] | 4-[6-(2-Chloro-4-fluoro-phenyl)-pyrimidin- 4-ylamino]-N-(3,4-dimethyl-isoxazol-5-yl)- benzenesulfonamide | C21H17ClFN5O3S |
| 20 | 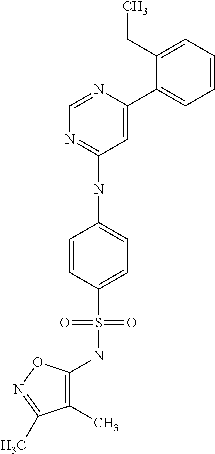 [SMILES: CCc1ccccc1-c1cc(Nc2ccc(S(=O)(=O)Nc3onc(C)c3C)cc2)ncn1] | N-(3,4-Dimethyl-isoxazol-5-yl)-4-[6-(2- ethyl-phenyl)-pyrimidin-4-ylamino]- benzenesulfonamide | C23H23N5O3S |
| 21 | 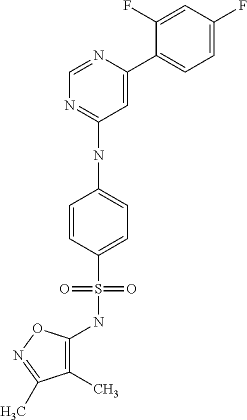 [SMILES: Cc1noc(NS(=O)(=O)c2ccc(Nc3cc(-c4ccc(F)cc4F)ncn3)cc2)c1C] | 4-[6-(2,4-Difluoro-phenyl)-pyrimidin-4- ylamino]-N-(3,4-dimethyl-isoxazol-5-yl)- benzenesulfonamide | C21H17F2N5O3S |
| 22 | 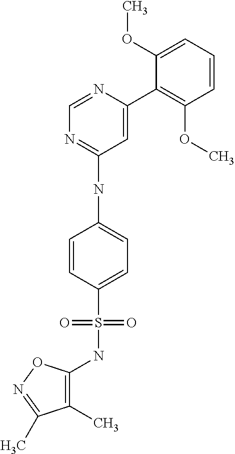 [SMILES: COc1cccc(OC)c1-c1cc(Nc2ccc(S(=O)(=O)Nc3onc(C)c3C)cc2)ncn1] | 4-[6-(2,6-Dimethoxy-phenyl)-pyrimidin-4- ylamino]-N-(3,4-dimethyl-isoxazol-5-yl)- benzenesulfonamide | C23H23N5O5S |
| 23 | 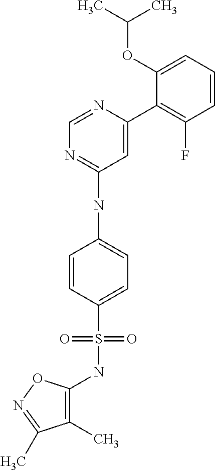 [SMILES: Cc1noc(NS(=O)(=O)c2ccc(Nc3cc(-c4c(F)cccc4OC(C)C)ncn3)cc2)c1C] | N-(3,4-Dimethyl-isoxazol-5-yl)-4-[6-(2- fluoro-6-isopropoxy-phenyl)-pyrimidin-4- ylamino]-benzenesulfonamide | C24H24FN5O4S |
| 24 | 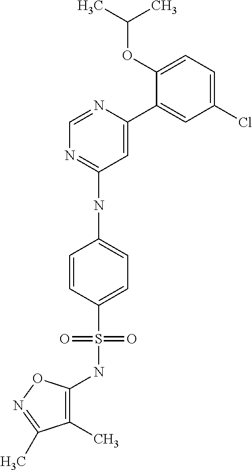 [SMILES: Cc1noc(NS(=O)(=O)c2ccc(Nc3cc(-c4cc(Cl)ccc4OC(C)C)ncn3)cc2)c1C] | 4-[6-(5-Chloro-2-isopropoxy-phenyl)- pyrimidin-4-ylamino]-N-(3,4-dimethyl- isoxazol-5-yl)-benzenesulfonamide | C24H24ClN5O4S |
| 25 | 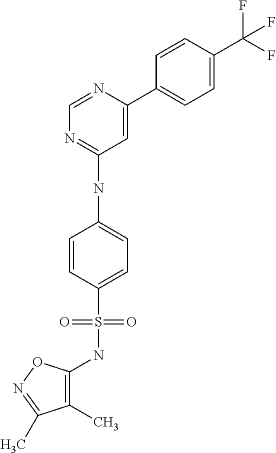 [SMILES: Cc1noc(NS(=O)(=O)c2ccc(Nc3cc(-c4ccc(C(F)(F)F)cc4)ncn3)cc2)c1C] | N-(3,4-Dimethyl-isoxazol-5-yl)-4-[6-(4- trifluoromethyl-phenyl)-pyrimidin-4- ylamino]-benzenesulfonamide | C22H18F3N5O3S |
| 26 | 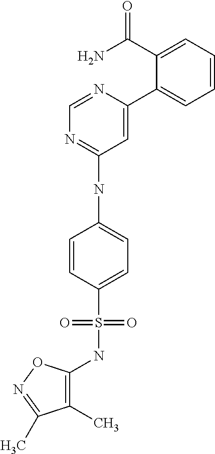 [SMILES: Cc1noc(NS(=O)(=O)c2ccc(Nc3cc(-c4ccccc4C(N)=O)ncn3)cc2)c1C] | 2-{6-[4-(3,4-Dimethyl-isoxazol-5- ylsulfamoyl)-phenylamino]-pyrimidin-4- yl}-benzamide | C22H20N6O4S |
| 27 | 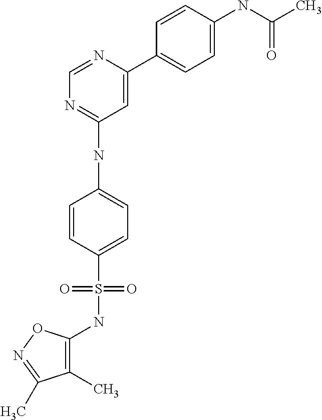 [SMILES: CC(=O)Nc1ccc(-c2cc(Nc3ccc(S(=O)(=O)Nc4onc(C)c4C)cc3)ncn2)cc1] | N-(4-{6-[4-(3,4-Dimethyl-isoxazol-5- ylsulfamoyl)-phenylamino]-pyrimidin-4- yl}-phenyl)-acetamide | C23H22N6O4S |
| 28 | 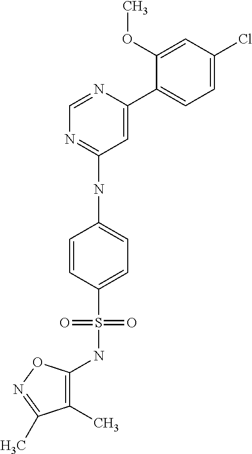 [SMILES: COc1cc(Cl)ccc1-c1cc(Nc2ccc(S(=O)(=O)Nc3onc(C)c3C)cc2)ncn1] | 4-[6-(4-Chloro-2-methoxy-phenyl)- pyrimidin-4-ylamino]-N-(3,4-dimethyl- isoxazol-5-yl)-benzenesulfonamide | C22H20ClN5O4S |
| 29 | 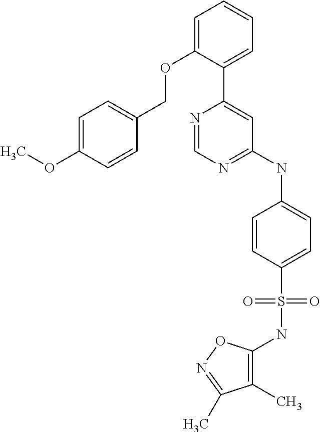 [SMILES: COc1ccc(COc2ccccc2-c2cc(Nc3ccc(S(=O)(=O)Nc4onc(C)c4C)cc3)ncn2)cc1] | N-(3,4-Dimethyl-isoxazol-5-yl)-4-{6-[2-(4- methoxy-benzyloxy)-phenyl]-pyrimidin-4- ylamino}-benzenesulfonamide | C29H27N5O5S |
| 30 | 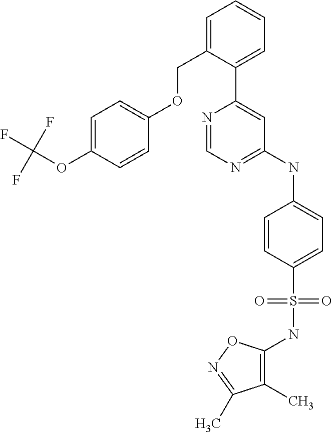 [SMILES: Cc1noc(NS(=O)(=O)c2ccc(Nc3cc(-c4ccccc4COc4ccc(OC(F)(F)F)cc4)ncn3)cc2)c1C] | N-(3,4-Dimethyl-isoxazol-5-yl)-4-{6-[2-(4- trifluoromethoxy-phenoxymethyl)-phenyl]- pyrimidin-4-ylamino}- benzenesulfonamide | C29H24F3N5O5S |
| 31 | 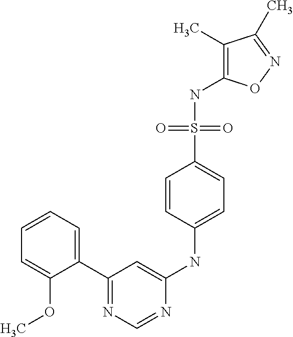 [SMILES: COc1ccccc1-c1cc(Nc2ccc(S(=O)(=O)Nc3onc(C)c3C)cc2)ncn1] | N-(3,4-Dimethyl-isoxazol-5-yl)-4-[6-(2- methoxy-phenyl)-pyrimidin-4-ylamino]- benzenesulfonamide | C22H21N5O4S |
| 32 | 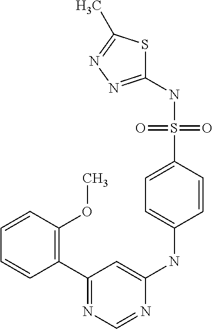 [SMILES: COc1ccccc1-c1cc(Nc2ccc(S(=O)(=O)Nc3nnc(C)s3)cc2)ncn1] | 4-[6-(2-Methoxy-phenyl)-pyrimidin-4- ylamino]-N-(5-methyl-[1,3,4]thiadiazol-2- yl)-benzenesulfonamide | C20H18N6O3S2 |
| 33 | 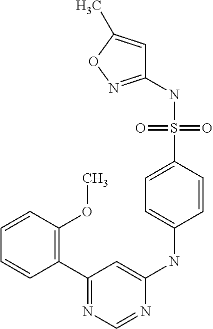 [SMILES: COc1ccccc1-c1cc(Nc2ccc(S(=O)(=O)Nc3cc(C)on3)cc2)ncn1] | 4-[6-(2-Methoxy-phenyl)-pyrimidin-4- ylamino]-N-(5-methyl-isoxazol-3-yl)- benzenesulfonamide | C21H19N5O4S |
| 34 | 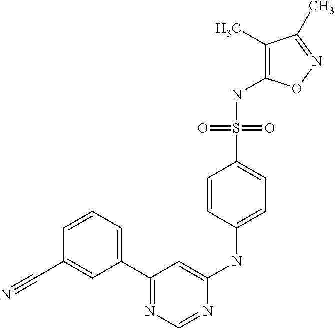 [SMILES: Cc1noc(NS(=O)(=O)c2ccc(Nc3cc(-c4cccc(C#N)c4)ncn3)cc2)c1C] | 4-[6-(3-Cyano-phenyl)-pyrimidin-4- ylamino]-N-(3,4-dimethyl-isoxazol-5-yl)- benzenesulfonamide | C22H18N6O3S |
| 35 | 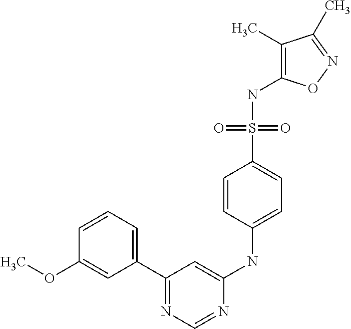 [SMILES: COc1cccc(-c2cc(Nc3ccc(S(=O)(=O)Nc4onc(C)c4C)cc3)ncn2)c1] | N-(3,4-Dimethyl-isoxazol-5-yl)-4-[6-(3- methoxy-phenyl)-pyrimidin-4-ylamino]- benzenesulfonamide | C22H21N5O4S |
| 36 | 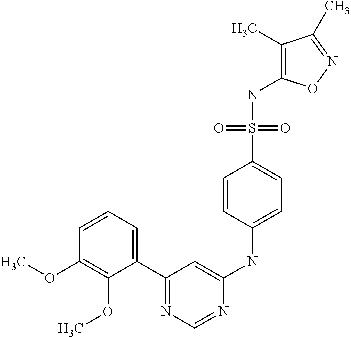 [SMILES: COc1cccc(-c2cc(Nc3ccc(S(=O)(=O)Nc4onc(C)c4C)cc3)ncn2)c1OC] | 4-[6-(2,3-Dimethoxy-phenyl)-pyrimidin-4- ylamino]-N-(3,4-dimethyl-isoxazol-5-yl)- benzenesulfonamide | C23H23N5O5S |
| 37 | 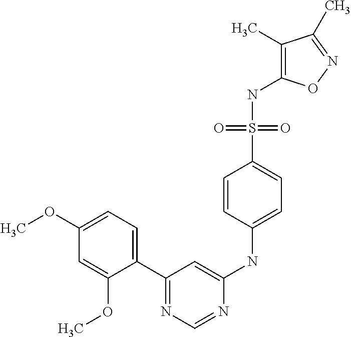 [SMILES: COc1ccc(-c2cc(Nc3ccc(S(=O)(=O)Nc4onc(C)c4C)cc3)ncn2)c(OC)c1] | 4-[6-(2,4-Dimethoxy-phenyl)-pyrimidin-4- ylamino]-N-(3,4-dimethyl-isoxazol-5-yl)- benzenesulfonamide | C23H23N5O5S |
| 38 | 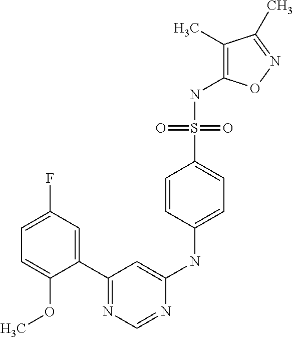 [SMILES: COc1ccc(F)cc1-c1cc(Nc2ccc(S(=O)(=O)Nc3onc(C)c3C)cc2)ncn1] | N-(3,4-Dimethyl-isoxazol-5-yl)-4-[6-(5- fluoro-2-methoxy-phenyl)-pyrimidin-4- ylamino]-benzenesulfonamide | C22H20FN5O4S |
| 39 | 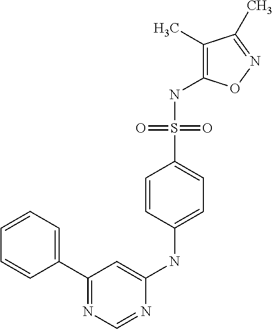 [SMILES: Cc1noc(NS(=O)(=O)c2ccc(Nc3cc(-c4ccccc4)ncn3)cc2)c1C] | N-(3,4-Dimethyl-isoxazol-5-yl)-4-(6- phenyl-pyrimidin-4-ylamino)- benzenesulfonamide | C21H19N5O3S |
| 40 | 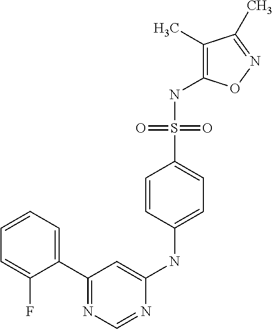 [SMILES: Cc1noc(NS(=O)(=O)c2ccc(Nc3cc(-c4ccccc4F)ncn3)cc2)c1C] | N-(3,4-Dimethyl-isoxazol-5-yl)-4-[6-(2- fluoro-phenyl)-pyrimidin-4-ylamino]- benzenesulfonamide | C21H18FN5O3S |
| 41 | 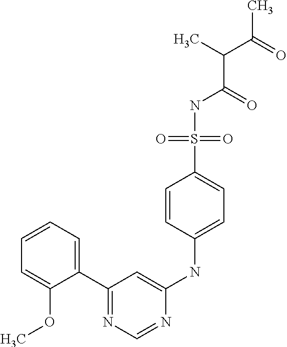 [SMILES: COc1ccccc1-c1cc(Nc2ccc(S(=O)(=O)NC(=O)C(C)C(C)=O)cc2)ncn1] | 4-[6-(2-Methoxy-phenyl)-pyrimidin-4- ylamino]-N-(2-methyl-3-oxo-butyryl)- benzenesulfonamide | C22H22N4O5S |
| 42 | 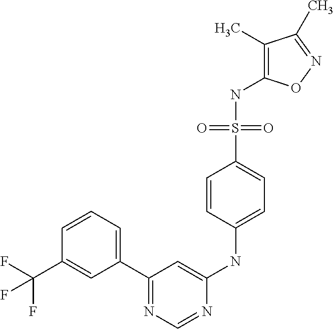 [SMILES: Cc1noc(NS(=O)(=O)c2ccc(Nc3cc(-c4cccc(C(F)(F)F)c4)ncn3)cc2)c1C] | N-(3,4-Dimethyl-isoxazol-5-yl)-4-[6-(3- trifluoromethyl-phenyl)-pyrimidin-4- ylamino]-benzenesulfonamide | C22H18F3N5O3S |
| 43 | 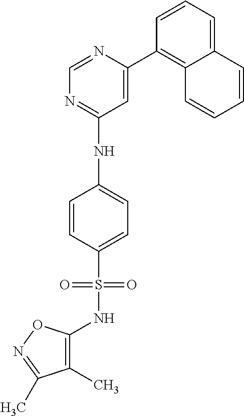 [SMILES: Cc1noc(NS(=O)(=O)c2ccc(Nc3cc(-c4cccc5ccccc45)ncn3)cc2)c1C] | N-(3,4-Dimethyl-isoxazol-5-yl)-4-(6- naphthalen-1-yl-pyrimidin-4-ylamino)- benzenesulfonamide | C25H21N5O3S |
| 44 | 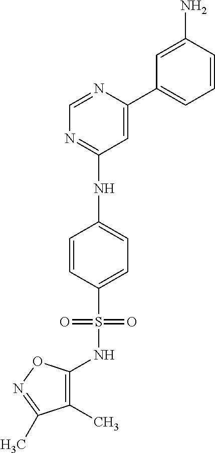 [SMILES: Cc1noc(NS(=O)(=O)c2ccc(Nc3cc(-c4cccc(N)c4)ncn3)cc2)c1C] | 4-[6-(3-Amino-phenyl)-pyrimidin-4- ylamino]-N-(3,4-dimethyl-isoxazol-5-yl)- benzenesulfonamide | C21H20N6O3S |
| 45 | 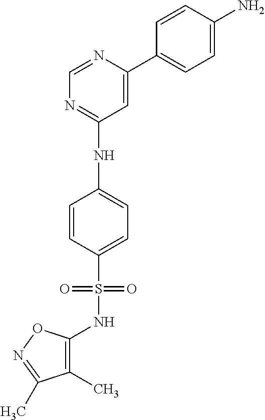 [SMILES: Cc1noc(NS(=O)(=O)c2ccc(Nc3cc(-c4ccc(N)cc4)ncn3)cc2)c1C] | 4-[6-(4-Amino-phenyl)-pyrimidin-4- ylamino]-N-(3,4-dimethyl-isoxazol-5-yl)- benzenesulfonamide | C21H20N6O3S |
| 46 | 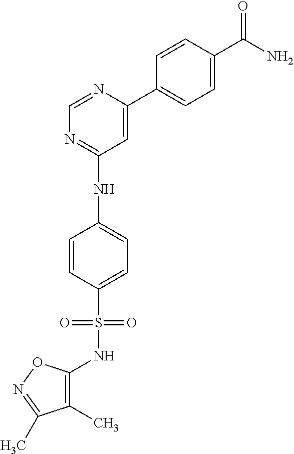 [SMILES: Cc1noc(NS(=O)(=O)c2ccc(Nc3cc(-c4ccc(C(N)=O)cc4)ncn3)cc2)c1C] | 4-{6-[4-(3,4-Dimethyl-isoxazol-5- ylsulfamoyl)-phenylamino]-pyrimidin-4- yl}-benzamide | C22H20N6O4S |
| 47 | 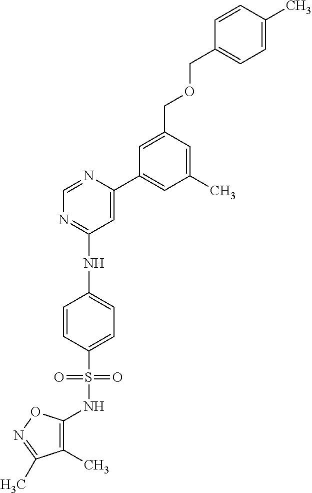 [SMILES: Cc1ccc(COCc2cc(C)cc(-c3cc(Nc4ccc(S(=O)(=O)Nc5onc(C)c5C)cc4)ncn3)c2)cc1] | N-(3,4-Dimethyl-isoxazol-5-yl)-4-{6-[3- methyl-5-(4-methyl-benzyloxymethyl)- phenyl]-pyrimidin-4-ylamino}- benzenesulfonamide | C31H31N5O4S |
| 48 | 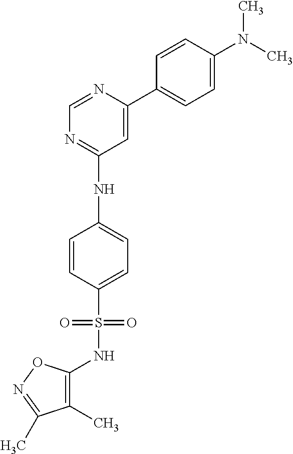 [SMILES: Cc1noc(NS(=O)(=O)c2ccc(Nc3cc(-c4ccc(N(C)C)cc4)ncn3)cc2)c1C] | 4-[6-(4-Dimethylamino-phenyl)-pyrimidin- 4-ylamino]-N-(3,4-dimethyl-isoxazol-5-yl)- benzenesulfonamide | C23H24N6O3S |

TABLE 2
The analytical data of the prepared compounds

| | | LCMS RT | | | |
|---|---|---|---|---|---|
| | | [min]/MS | MW calc. | | |
| Example | 1H-NMR | method | monoisotopic | MH− | MH+ |
| 1 | 10.89 (bs, 1H), 10.23 (s, 1H), 8.81 (s, | 3.81 | 489.1 | 487.91; | 489.96; |
| | 1H), 7.97 (d, J = 8.85 Hz, 2H), 7.89 (d, | B | | 487.89 | 490.15 |
| | J = 7.62 HZ, 1H), 7.81 (t, J = 7.29 Hz, | | | | |
| | 1H), 7.72 (m, 3H), 7.60 (d, J = 7.41 Hz, | | | | |
| | 1H), 6.92 (s, 1H), 2.09 (s, 3H), 1.64 (s, | | | | |
| | 3H). | | | | |
| 2 | 10.95 (bs, 1H), 10.27 (s, 1H), 8.86 (s, | 4.04 | 505.1 | 503.94 | 505.98 |
| | 1H), 7.97 (d, J = 8.76 Hz, 2H), 7.89 (d, | A | | | |
| | J = 7.56 Hz, 1H), 7.73 (d = 8.73 Hz, | | | | |
| | 2H), 7.60 (m, 3H), 7.23 (s, 1H), 2.09 | | | | |
| | (s, 3H), 1.65 (s, 3H). | | | | |
| 3 | 10.87 (bs, 1H), 10.29 (s, 1H), 8.82 (s, | | | | |
| | 1H), 7.99 (d, J = 8.64 Hz, 2H), 7.78 (d, | | | | |
| | J = 8.01 Hz, 1H), 7.72 (d, J = 8.67 Hz, | 3.80 | 465.1 | 463.92 | 465.96 |
| | 2H), 7.60 (s, 1H), 6.23 (s, 2H), 2.09 (s, | A | | | |
| | 3H), 1.66 (s, 3H). | | | | |
| 4 | 10.86 (bs, 1H), 10.15 (s, 1H), 8.81 (s, | | | | |
| | 1H), 8.06 (d, J = 6.75 Hz, 1H), 7.91 (d, | | | | |
| | J = 8.79 Hz, 2H), 7.69 (d, J = 8.76 Hz, | | | | |
| | 2H), 7.55 (s, 1H), 7.49 (t,, J = 7.14 Hz, | | | | |
| | 1H), 7.40 (t,, J = 7.89 Hz, 2H), 7.31 (t,, | 4.12 | 513.1 | 511.97 | 514.02 |
| | J = 7.56 Hz, 1H), 7.14 (t,, J = 7.32 Hz, | A | | | |
| | 1H), 7.04 (d, J = 8.16 Hz, 2H), 6.98 (d, | | | | |
| | J = 8.16 Hz, 1H), 2.08 (s, 3H), 1.64 (s, | | | | |
| | 3H). | | | | |
| 5 | 10.95 (bs, 1H), 10.12 (s, 1H), 8.81 (s, | 3.79 | 493.2 | 492 | 494.04 |
| | 1H), 7.97 (d, J = 8.73 Hz, 2H), 7.86 (d, | A | | | |
| | J = 1.62 Hz, 1H), 7.73 (d, J = 8.70 Hz, | | | | |
| | 2H), 7.56 (s, 1H), 7.34 (dd, J1 = 8.37 | | | | |
| | Hz, J2 = 1.68 Hz, 1H), 7.12 (d, J = 8.55 | | | | |
| | Hz, 1H), 3.89 (s, 3H), 2.92 (m, 1H), | | | | |
| | 2.09 (s, 3H), 1.65 (s, 3H), 1.21 (d, | | | | |
| | J = 6.87 Hz, 6H). | | | | |
| 6 | 10.96 (bs, 1H), 10.12 (s, 1H), 8.82 (s, | 3.95 | 507.2 | 506.04 | 508.08 |
| | 1H), 8.02 (d, J = 2.46 Hz, 1H), 7.97 (d, | A | | | |
| | J = 8.82 Hz, 2H), 7.72 (d, J = 8.79 Hz, | | | | |
| | 2H), 7.56 (s, 1H), 7.49 (dd, J1 = 8.67 | | | | |
| | Hz, J2 = 2.52 Hz, 1H), 7.12 (d, J = 8.73 | | | | |
| | Hz, 1H), 3.89 (s, 3H), 2.09 (s, 3H), | | | | |
| | 1.66 (s, 3H), 1.30 (s, 9H). | | | | |
| 7 | 10.86 (bs, 1H), 10.14 (s, 1H), 8.88 (s, | 3.31 | 481.14 | 479.96 | 482.02 |
| | 1H), 7.97 (d, J = 8.76 Hz, 2H), 7.72 (d, | A | | | |
| | J = 8.73 Hz, 2H), 7.61 (s, 1H), 7.58 (d, | | | | |
| | J = 3.03 Hz, 1H), 7.15 (d, J = 9.00 Hz, | | | | |
| | 1H), 7.05 (dd, J1 = 8.97 Hz, J2 = 3.13 | | | | |
| | Hz, 1H), 3.87 (s, 3H), 3.77 (s, 3H), | | | | |
| | 2.09 (s, 3H), 1.66 (s, 3H). | | | | |
| 8 | 10.87 (bs, 1H), 10.08 (s, 1H), 8.80 (s, | 3.37 | 465.15 | 463.94 | 466 |
| | 1H), 7.95 (m, 3H), 7.72 (d, J = 8.64 Hz, | A | | | |
| | 2H), 7.58 (s, 1H), 7.45 (t,, J = 7.26 Hz, | | | | |
| | 1H), 7.18 (d, J = 8.31 Hz, 1H), 7.06 (t, | | | | |
| | J = 6.87 Hz, 4.18 (m, 2H), 2.09 (s, 3H), | | | | |
| | 1.66 (s, 3H), 1.41 (t, J = 6.87 Hz, 3H). | | | | |
| 9 | 10.86 (bs, 1H), 10.11 (s, 1H), 8.79 (s, | 3.25 | 465.15 | 463.94; | 456.97; |
| | 1H), 7.97 (d, J = 8.82 Hz, 2H), 7.80 (d, | B | | 464.14 | 466.38 |
| | J = 1.89 Hz, 1H), 7.72 (d, J = 8.76 Hz, | | | | |
| | 2H), 7.56 (s, 1H), 7.27 (dd, J1 = 8.37 | | | | |
| | Hz, J2 = 1.95 Hz, 1H), 7.09 (d, J = 8.43 | | | | |
| | Hz, 1H), 3.88 (s, 3H), 2.31 (s, 3H), | | | | |
| | 2.08 (s, 3H), 1.65 (s. 3H) | | | | |
| 10 | 10.88 (bs, 1H), 10.06 (s, 1H), 8.80 (s, | 3.60 | 479.6 | 478 | 480.04 |
| | 1H), 7.95 (m, 3H), 7.72 (d, J = 8.52 Hz, | A | | | |
| | 2H), 7.54 (s, 1H), 7.44 (t, J = 7.44 Hz, | | | | |
| | 1H), 7.18 (d, J = 8.25 Hz, 1H), 7.08 (t, | | | | |
| | J = 7.20 Hz, 1H), 4.07 (m, 2H), 2.08 (s, | | | | |
| | 3H), 1.80 (m, 2H), 1.66 (s, 3H), 0.95 | | | | |
| | (t,J = 7.23 Hz, 3H). | | | | |
| 11 | 10.87 (bs, 1H), 10.10 (s, 1H), 8.77 (s, | 3.59 | 465.11 | 463.91 | 465.96 |
| | 1H), 7.95 (d, J = 8.82 Hz, 2H), 7.72 (d, | A | | | |
| | J = 8.79 Hz, 2H), 7.62 (d, J = 1.32 Hz, | | | | |
| | 1H), 7.24 (s, 1H), 7.08 (d, J = 8.16 Hz, | | | | |
| | 1H), 6.16 (s, 2H), 209 (s, 3H), 1.66 (s, | | | | |
| | 3H). | | | | |
| 12 | 10.88 (bs, 1H), 10.14 (s, 1H), 8.80 (s, | 3.19 | 469.12 | 467.93 | 469.97 |
| | 1H), 7.95 (d, J = 8.70 Hz, 2H), 7.73 (d, | A | | | |
| | J = 8.61 Hz, 2H), 7.47 (m, 1H), 7.02 (d, | | | | |
| | J = 8.58 Hz, 1H), 6.93 (m, 2H), 3.79 (s, | | | | |
| | 3H), 2.09 (s, 3H), 1.65 (s, 3H). | | | | |
| 13 | 10.87 (bs, 1H), 10.06 (s, 1H), 8.79 (s, | 3.54 | 479.16 | 477.96 | 480.03 |
| | 1H), 7.94 (m, 3H), 7.72 (d, J = 8.64 Hz, | A | | | |
| | 2H), 7.56 (s, 1H), 7.43 (t,, J = 7.32 Hz, | | | | |
| | 1H), 7.19 (d, J = 8.28 Hz, 1H), 7.06 (t,, | | | | |
| | J = 7.44 Hz, 1H), 4.73 (m, 1H), 2.09 (s, | | | | |
| | 3H), 1.66 (s, 3H), 1.33 (d, J = 5.88 Hz, | | | | |
| | 6H). | | | | |
| 14 | 10.89 (bs, 1H), 10.19 (s, 1H), 8.82 (s, | 3.53 | 487.11 | 485.92 | 487.98 |
| | 1H), 7.95 (d, J = 8.64 Hz, 2H), 7.73 (d, | A | | | |
| | J = 8.58 Hz, 2H), 7.53 (m, 1H), 6.99 (m, | | | | |
| | 2H), 3.78 (s, 3H), 2.09 (s, 3H), 1.65 (s, | | | | |
| | 3H). | | | | |
| 15 | 10.88 (bs, 1H), 10.13 (s, 1H), 8.82 (s, | 3.45 | 485.09 | 483.92 | 485.96 |
| | 1H), 7.95 (d, J = 8.79 Hz, 2H), 7.73 (d, | A | | | |
| | J = 8.73 Hz, 2H), 7.46 (t, J = 8.25 Hz, | | | | |
| | 1H), 7.16 (m, 2H), 6.82 (s, 1H), 3.77 | | | | |
| | (s, 3H), 2.09 (s, 3H), 1.65 (s, 3H). | | | | |
| 16 | 10.89 (bs, 1H), 10.29 (s, 1 H), 8.85 (s, | 4.20 | 489.04 | 487.84 | 489.9 |
| | 1H), 7.97 (d, J = 7.89 Hz, 2H), 7.67 (m, | A | | | |
| | 5H), 7.23 (s, 1H), 2.09 (s, 3H), 1.65 (s, | | | | |
| | 3H). | | | | |
| 17 | 10.87 (bs, 1H), 10.06 (s, 1H), 8.78 (s, | 4.00 | 497.15 | 495.96 | 498.01 |
| | 1H), 8.02 (t, J = 8.46 Hz, 1H), 7.95 (d, | A | | | |
| | J = 8.79 Hz, 2H), 7.72 (d, J = 8.79 Hz, | | | | |
| | 2H), 7.54 (s, 1H), 7.11 (dd, J1 = 11.64 | | | | |
| | Hz, J2 = 1.98 Hz, 1H), 6.89 (dt,, | | | | |
| | J1 = 8.52 Hz, J2 = 2.22 Hz, 1H) 4.79 | | | | |
| | (m, 1H), 2.09 (s, 3H), 1.66 (s, 3H), 1.35 | | | | |
| | (d, J = 5.97 Hz, 6H). | | | | |
| 18 | 10.88 (bs, 1H), 10.12 (s, 1H), 8.79 )s, | 3.55 | 469.12 | 467.94 | 469.94 |
| | 1H), 8.06 (t, J = 8.25 Hz, 1H), 7.96 (d, | A | | | |
| | J = 8.79 Hz, 2H), 7.72 (d, J = 8.76 Hz, | | | | |
| | 2H), 7.55 (s, 1H), 7.11 (dd, J1 = 11.49 | | | | |
| | Hz, J2 = 2.16 Hz, 1H), 6.96 (dt,, | | | | |
| | J1 = 8.37 Hz, J2 = 2.10 Hz, 1H), 3.94 | | | | |
| | (s, 3H), 2.08 (s, 3H), 1.65 (s, 3H). | | | | |
| 19 | 10.89 (bs, 1H), 10.26 (s, 1H), 8.84 (s, | 3.97 | 473.07 | 471.83 | 473.89 |
| | 1H), 7.97 (d, J = 8.70 Hz, 2H (, 7.75 (m, | A | | | |
| | 3H), 7.60 (dd, J1 = 8.88 Hz, J2 = 2.28 | | | | |
| | Hz, 1H), 7.39 (d,, J1 = 8.46 Hz, | | | | |
| | J2 = 2.40 Hz, 1H), 7.20 (s, 1H), 2.09 (s, | | | | |
| | 3H), 1.65 (s, 3H). | | | | |
| 20 | 10.88 (bs, 1H), 10.13 (s, 1H), 8.81 (s, | 3.76 | 449.15 | 448.09 | 450.13 |
| | 1H), 7.97 (d, J = 8.73 Hz, 2H), 7.73 (d, | A | | | |
| | J = 8.70 Hz, 2H), 7.39 (m, 4H), 6.95 (s, | | | | |
| | 1H), 2.74 (m, 2H), 2.09 (s, 3H), 1.65 | | | | |
| | (s, 3H), 1.11 (t,, J = 7.41 Hz, 3H). | | | | |
| 21 | 10.88 (bs, 1H), 10.26 (s, 1H), 8.85 (s, | 3.98 | 457.1 | 456.01 | 458.05 |
| | 1H), 8.20 (m, 1H), 7.98 (d, J = 8.76 Hz, | A | | | |
| | 2H), 7.73 (d, J = 8.73 Hz, 2H), 7.45 (m, | | | | |
| | 1H), 7.37 (s, 1H), 7.28 (dt,, J1 = 8.64 | | | | |
| | Hz, J2 = 2.43 Hz, 1H), 2.09 (s, 3H), | | | | |
| | 1.66 (s, 3H). | | | | |
| 22 | 10.87 (bs, 1H), 10.01 (s, 1H), 8.76 (s, | 3.00 | 481.12 | 480.08 | 482.11 |
| | 1H), 7.94 (d, J = 8.82 Hz, 2H), 7.71 (d, | A | | | |
| | J = 8.85 Hz, 2H), 7.38 (m, 1H), 6.77 (m, | | | | |
| | 3H), 3.69 (s, 6H), 2.08 (s, 3H), 1.64 (s, | | | | |
| | 3H). | | | | |
| 23 | 10.88 (bs, 1H), 10.12 (s, 1H), 8.80 (s, | 3.60 | 497.15 | 496.09 | 498.13 |
| | 1H), 7.96 (d, J = 8.58 Hz, 2H), 7.73 (d, | A | | | |
| | J = 8.55 Hz, 2H), 7.43 (m, 1H), 7.03 (d, | | | | |
| | J = 8.31 Hz, 1H), 6.94 (s, 1H), 6.87 (d, | | | | |
| | J = 8.58 Hz, 1H), 4.63 (m, 1H), 2.09 (s, | | | | |
| | 1H), 1,65 (s, 3H), 1.20 (d, J = 5.91 Hz, | | | | |
| | 6H). | | | | |
| 24 | 10.85 (bs, 1H), 10.13 (s, 1H), 8.80 (s, | 4.39 | 513.12 | 512.08 | 514.11 |
| | 1H), 7.96 (m, 3H), 7.73 (d, J = 8.46 Hz, | A | | | |
| | 2H), 7.59 (s, 1H), 7.48 (d, J = 8.43 Hz, | | | | |
| | 1H), 7.24 (d, J = 8.82 Hz, 1H), 4.75 (m, | | | | |
| | 1H), 2.09 (s, 3H), 1.67 (s, 3H), 1.33 | | | | |
| | (d, J = 5.76 Hz, 6H). | | | | |
| 25 | 10.90 (bs, 1H), 10.27 (s, 1H), 8.87 (s, | 4.34 | 489.11 | 488.1 | 490.11 |
| | 1H), 8.25 (d, J = 7.62 Hz, 2H), 7.98 (d, | A | | | |
| | J = 8.52 Hz, 2H), 7.92 (d, J = 7.83 Hz, | | | | |
| | 2H), 7.74 (d, J = 8.22 Hz, 2H), 7.43 (s, | | | | |
| | 1H), 2.09 (s, 3H), 1.66 (s, 3H). | | | | |
| 26 | 10.87 (bs, 1H), 10.18 (s, 1H), 8.76 (s, | 2.75 | 464.13 | 463.09 | 465.11 |
| | 1H), 7.94 (d, J = 8.52 Hz, 2H), 7.83 (bs, | A | | | |
| | 1H), 7.73 (d, J = 8.67 Hz, 2H), 7.63 (m, | | | | |
| | 1H), 7.56 (m, 2H), 7.35 (bs, 1H), 7.06 | | | | |
| | (s, 1H), 2.09 (s, 3H), 1.65 (s, 3H). | | | | |
| 27 | 10.87 (bs, 1H), 10.16 (s, 1H), 10.13 | 3.05 | 478.14 | 477.15 | 479.17 |
| | (s, 1H), 8.78 (s, 1H), 8.02 (d, J = 8.46 | A | | | |
| | Hz, 2H), 7.96 (d, J = 8.82 Hz, 2H), 7.75 | | | | |
| | (d, J = 9.03 Hz, 2H), 7.72 (s, J = 8.94 | | | | |
| | Hz, 2H), 7.29 (s, 1H), 2.09 (s, 3H), | | | | |
| | 1.66 (s, 3H). | | | | |
| 28 | 10.89 (bs, 1H), 10.15 (s, 1H), 8.80 (s, | 3.82 | 485.09 | 484.14 | 486.18 |
| | 1H), 8.02 (d, J = 8.41 Hz, 1H), 7.97 (d, | A | | | |
| | J = 8.79 Hz, 2H), 7.72 (d, J = 8.76 Hz, | | | | |
| | 2H), 7.56 (s, 1H), 7.30 (s, 1H), 7.14 | | | | |
| | (dd, J1 = 8.34 Hz, J2 = 1.53 Hz, 1H), | | | | |
| | 3.96 (s, 3H), 2.09 (s, 3H), 1.66 )s, 3H). | | | | |
| 29 | 10.90 (bs, 1H), 10.08 (s, 1H), 8.81 (s, | 3.72 | 557.13 | 556.16 | 558.15 |
| | 1H), 7.90 (m, 3H), 7.71 (d, J = 8.46 Hz, | B | | | |
| | 2H), 7.56 (s, 1H), 7.43 (d, J = 7.77 Hz, | | | | |
| | 1H), 7.37 (d, J = 8.37 Hz, 2H, 7.23 (d, | | | | |
| | J = 8.07 Hz, 1H), 7.08 (t,, J = 7.50 Hz, | | | | |
| | 1H), 6.88 (d, J = 8.04 Hz, 2H), 5.21 (s, | | | | |
| | 2H), 3.71 (s, 3H), 2.08 (s, 3H), 1.65 (s, | | | | |
| | 3H). | | | | |
| 30 | 10.90 (bs, 1H), 10.15 (s, 1H), 8.75 (s, | 4.58 | 611.14 | 610.1 | 612.1 |
| | 1H), 7.94 (d, J = 8.64 Hz, 2H), 7.72 (d, | B | | | |
| | J = 8.70 Hz, 2H), 7.65 (m, 1H), 7.54 (m, | | | | |
| | 3H), 7.24 (d, J = 8.49 Hz, 2H), 7.05 (s, | | | | |
| | 1H), 6.97 (d, J = 8.94 hz, 2H), 5.35 (s, | | | | |
| | 2H), 2.08 (s, 3H), 1.65 (s, 3H). | | | | |
| 31 | 10.86 (bs, 1H), 10.12 (s, 1H), 8.80 (s, | 3.04 | 451.13 | 449.94 | 452.15 |
| | 1H), 7.98 (m, 3H), 7.71 (d, J = 8.73 Hz, | B | | | |
| | 2H), 7.57 (s, 1H), 7.48 (t, J = 7.26 Hz, | | | | |
| | 1H), 7.20 (d, J = 8.25 Hz, 1H), 7.10 (t, | | | | |
| | J = 7.50 Hz, 1H), 3.92 (s, 3H), 2.09 (s, | | | | |
| | 3H), 1.65 (s, 3H) | | | | |
| 32 | 13.84 (bs, 1H), 10.04 (s, 1H), 8.77 (s, | 2.78 | 454.08 | 452.91 | 454.93 |
| | 1H), 7.98 (dd, J³ = 7.65 Hz, J⁴ = 1.26 | A | | | |
| | Hz, 1H), 7.92 (d, J = 8.16 Hz, 2H), 7.75 | | | | |
| | (d, J = 8.73 Hz, 2H), 7.54 (s, 1H), 7.47 | | | | |
| | (t, J = 8.46 Hz, 1H), 7.19 (d, J = 8.31 | | | | |
| | Hz, 1H), 7.08 (t, J = 7.47 Hz, 1H), 3.91 | | | | |
| | (s, 3H), 2.45 (s, 3H) | | | | |
| 33 | 11.26 (bs, 1H), 10.10 (s, 1H), 8.79 (s, | 3.09 | 437.12 | 437.12 | 435.91 |
| | 1H), 7.96 (m, 3H), 7.81 (d, J = 8.82 Hz, | A | | | |
| | 2H), 7.55 (s, 1H), 7.47 (t, J = 8.55 Hz, | | | | |
| | 1H), 7.19 (d, J = 8.31 Hz, 1H), 7.09 (t, | | | | |
| | J = 7.44 Hz, 1H), 6.15 (s, 1H), 3.91 (s, | | | | |
| | 3H), 2.30 (s, 3H) | | | | |
| 34 | 10.89 (bs, 1H), 10.24 (s, 1H), 8.86 (s, | 3.80 | 446.11 | 445.04 | 447.1 |
| | 1H), 8.45 (s, 1H), 8.36 (d, J = 7.95 Hz, | A | | | |
| | 1H), 7.99 (m, 3H), 7.77 (m, 3H), 7.41 | | | | |
| | (s, 1H), 2.09 (s, 3H), 1.66 (s, 3H) | | | | |
| 35 | 10.88 (bs, 1H), 10.17 (s, 1H), 8.83 (s, | 3.71 | 451.13 | 450.08 | 452.11 |
| | 1H), 7.97 (d, J = 8.79 Hz, 2H), 7.73 (d, | A | | | |
| | J = 8.73 Hz, 2H), 7.61 (m, 2H), 7.47 (t, | | | | |
| | J = 8.16 Hz, 1H), 7.35 (s, 1H), 7.11 (dd, | | | | |
| | J³ = 7.62 Hz, J⁴ = 1.74 Hz, 1H), 3.85 (s, | | | | |
| | 3H), 2.09 (s, 3H), 1.66 (s, 3H) | | | | |
| 36 | 10.87 (bs, 1H), 10.17 (s, 1H), 8.82 (s, | 3.21 | 481.14 | 480.04 | 482.29 |
| | 1H), 7.97 (d, J = 8.67 Hz, 2H), 7.72 (d, | B | | | |
| | J = 8.67 Hz, 2H), 7.46 (m, 2H), 7.19 (d, | | | | |
| | J = 4.53 Hz, 2H), 3.88 (s, 3H), 3.75 (s, | | | | |
| | 3H), 2.09 (s, 3H), 1.65 (s, 3H) | | | | |
| 37 | 10.86 (bs, 1H), 10.05 (s, 1H), 8.75 (s, | 3.16 | 481.14 | 480.12 | 482.16 |
| | 1H), 8.05 (d, J = 8.55 Hz, 1H), 7.96 (d, | A | | | |
| | J = 8.76 Hz, 2H), 7.71 (d, J = 8.73 Hz, | | | | |
| | 2H), 7.57 (s, 1H), 6.70 (m, 2H), 3.93 | | | | |
| | (s, 3H), 3.85 (s, 3H), 2.09 (s, 3H), 1.65 | | | | |
| | (s, 3H) | | | | |
| 38 | 10.88 (bs, 1H), 10.18 (s, 1H), 8.82 (s, | 3.67 | 469.12 | 468.11 | 470.11 |
| | 1H), 7.97 (d, J = 8.73 Hz, 2H), 7.80 (dd, | A | | | |
| | J³ = 9.87 Hz, J⁴ = 3.06 Hz, 1H), 7.72 (d, | | | | |
| | J = 8.76 Hz, 2H), 7.65 (s, 1H), 7.33 (m, | | | | |
| | 1H), 7.23 (m, 1H), 3.92 (s, 3H), 2.09 | | | | |
| | (s, 3H), 1.65 (s, 3H) | | | | |
| 39 | 10.89 (bs, 1H), 10.18 (s, 1H), 8.83 (s, | 3.61 | 421.12 | 420.09 | 422.14 |
| | 1H), 8.07 (m, 2H), 7.98 (d, J = 8.70 Hz, | A | | | |
| | 2H), 7.73 (d, J = 8.70 Hz, 2H) 7.55 (m, | | | | |
| | 3H), 7.35 (s, 1H), 2.09 (s, 3H), 1.66 (s, | | | | |
| | 3H) | | | | |
| 40 | 10.90 (bs, 1H), 10.26 (s, 1H), 8.86 (s, | 3.76 | 439.11 | 438.05 | 440.1 |
| | 1H), 8.13 (t, J = 7.77 Hz, 1H), 7.99 (d, | A | | | |
| | J = 8.82 Hz, 2H), 7.73 (d, J = 8.79 Hz, | | | | |
| | 2H), 7.57 (m, 1H), 7.40 (m, 3H), 2.09 | | | | |
| | (s, 3H), 1.66 (s, 3H) | | | | |
| 41 | 12.21 (bs, 1H), 10.16 (s, 1H), 8.81 (s, | 2.86 | 454.13 | 453.09 | 455.13 |
| | 1H), 7.99 (m, 2H), 7.88 (d, J = 8.79 Hz, | A | | | |
| | 2H), 7.56 (m, 3H), 7.20 (d, J = 8.22 Hz, | | | | |
| | 1H), 7.10 (t, J = 7.50 Hz, 1H), 3.92 (s, | | | | |
| | 3H), 3.58 (q, 1H), 2.04 (s, 3H), 1.10 (t, | | | | |
| | J = 2.94 Hz, 3H) | | | | |
| 42 | 10.99 (bs, 1H), 10.27 (s, 1H), 8.87 (s, | 4.35 | 489.11 | 488.14 | 490.18 |
| | 1H), 8.37 (s, 1H), 8.34 (d, J = 8.31 Hz, | A | | | |
| | 1H), 7.99 (d, J = 8.79 Hz, 2H), 7.92 (d, | | | | |
| | J = 7.77 Hz, 1H), 7.82 (t, J = 7.71 Hz, | | | | |
| | 1H), 7.74 (d, J = 8.79 Hz, 2H), 7.45 (s, | | | | |
| | 1H), 2.09 (s, 3H), 1.66 (s, 3H) | | | | |
| 43 | 10.92 (bs, 1H), 10.25 (s, 1H), 8.88 (s, | 4.20 | 471.1365 | 470.09 | 472.12 |
| | 1H), 8.68 (s, 1H), 8.14 (m, 4H), 7.99 | A | | | |
| | (d, J = 8.46 Hz, 2H), 7.74 (d, J = 8.67 | | | | |
| | Hz, 2H), 7.61 (m, 2H), 7.51 (s, 1H), | | | | |
| | 2.09 (s, 3H), 1.66 (s, 3H) ppm. | | | | |
| 44 | 10.14 (s, 1H), 8.78 (s, 1H), 7.96 (d, | 2.90 | 436.1318 | 435.06 | 437.09 |
| | J = 8.79 hz, 2H), 7.72 (d, J = 8.76 Hz, | A | | | |
| | 2H), 7.31 (s, 1H), 7.24 (s, 1H), 7.16 | | | | |
| | (bs, 2H), 6.70 (m, 1H), 2.09 (s, 3H), | | | | |
| | 1.65 (s, 3H) ppm. | | | | |
| 45 | 9.97 (s, 1H), 8.68 (s, 1H), 7.94 (d, | 2.80 | 436.1318 | 435.09 | 437.11 |
| | J = 8.67 Hz, 2H), 7.79 (d, J = 8.43 Hz, | A | | | |
| | 2H), 7.70 (d, J = 8.73 Hz, 2H), 7.13 (s, | | | | |
| | 1H), 6.66 (d, J = 8.43 Hz, 2H), 5.80 (bs, | | | | |
| | 3H), 2.08 (s, 3H), 1.65 (s, 3H) ppm. | | | | |
| 46 | 10.89 (bs, 1H), 10.22 (s, 1H), 8.85 (s, | 2.91 | 464.1267 | 463.17 | 465.16 |
| | 1H), 8.12 (d, J = 8.19 Hz, 2H), 8.07 (s, | B | | | |
| | 1H), 8.03 (d, J = 8.31 Hz, 2H), 7.97 (d, | | | | |
| | J = 8.67 Hz, 2H), 7.73 (d, J = 8.67 Hz, | | | | |
| | 2H), 7.46 (bs, 1H), 7.40 (s, 1H), 2.09 | | | | |
| | (s, 3H), 1.66 (s, 3H) ppm. | | | | |
| 47 | 10.87 (bs, 1H), 10.14 (s, 1H), 8.81 (s, | 4.74 | 569.2097 | 568.30 | 570.30 |
| | 1H), 7.97 (d, J = 8.16 Hz, 2H), 7.82 (d, | B | | | |
| | J = 8.31 Hz, 2H), 7.72 (d, J = 8.31 Hz, | | | | |
| | 2H), 7.26 (m, 6H), 4.57 (s, 2H), 4.53 | | | | |
| | (s, 3H), 2.41 (s, 3H), 2.30 (s, 3H), 2.09 | | | | |
| | (s, 3H), 1.66 (s, 3H) ppm. | | | | |
| 48 | 10.86 (bs, 1H), 10.00 (s, 1 H), 8.71 (s, | 3.00 | 464.1631 | 463.21 | 465.26 |
| | 1H), 7.93 (m, 4H), 7.70 (d, J = 8.76 Hz, | B | | | |
| | 2H), 7.19 (s, 1H), 6.83 (d, J = 8.82 Hz, | | | | |
| | 2H), 3.01 (s, 6H), 2.09 (s, 3H), 1.66 (s, | | | | |
| | 3H) ppm. | | | | |

### Biological Results

In Vitro PLK1 Assay

The activity of the compounds described in the present invention was determined using a commercially available IMAP Screening Express Assay Kit (Molecular devices).

This method measures the change in the fluorescent polarization of a fluorescently-labeled peptide due to the effect of human PLK1 kinase domain on it. PLK1 kinase assays were performed in low protein binding 384-well plates (Corning 3676). Test compounds were diluted in 100% DMSO to 5 mM stock concentration, the further dilutions were made in H₂O or 100% DMSO to desirable concentrations.

Each reaction consisted of 30 nM enzyme PLK1 kinase domain, 400 nM 5TAMRA-RGSFNDTLDFD-NH2 (Genecust Europe), 16 μM ATP (=Kmₐₚₚ, Sigma-Aldrich) and kinase buffer: 20 mM HEPES pH 7.5 (Sigma-Aldrich), 1 mM DTT (Sigma-Aldrich), 10 mM MgCl₂ (Sigma-Aldrich), 0.01% Triton X-100 (Sigma-Aldrich).

For each reaction, 4 or 6 μl containing 5TAMRA-RGSFNDTLDFD-NH2, ATP and kinase buffer were combined with 2 μl diluted compound in H₂O or 0.04 μl compound in 100% DMSO. The kinase reaction was started by the addition of 2 μl diluted enzyme. The reaction was allowed to run for 90 minutes at room temperature. The reaction was stopped by adding 15 μl IMAP beads (1:1200 beads in progressive (40% buffer A, 60% buffer B) 1× buffer). After an additional 60 minutes, fluorescent polarization (Ex: 550-10 nm, Em: 590-10 nm, Dich: 561 nm) was measured using Analyst GT multimode reader (Molecular Devices).

TABLE 3
The PLK1 inhibition data and IC50 values of the compounds.

| | PLK1 IC50 | PLK1 inhibition |
|---|---|---|
| Example | [uM] | [% at 10 uM] |
| 1 | 1.447 | 96.1 |
| 2 | 0.698 | 69.0 |
| 3 | | 48.5 |
| 4 | | 52.7 |
| 5 | | 43.2 |
| 6 | | 26.1 |
| 7 | 0.189 | 59.6 |
| 8 | 1.247 | 82.4 |
| 9 | 0.807 | 67.6 |
| 10 | 0.925 | 104.4 |
| 11 | 0.888 | 74.4 |
| 12 | 1.456 | 83.3 |
| 13 | 1.824 | 88.6 |
| 14 | | 55.4 |
| 15 | | 96.4 |
| 16 | 0.604 | 7.9 |
| 17 | 0.179 | 68.8 |
| 18 | 0.040 | 63.3 |
| 19 | 0.047 | 60.8 |
| 20 | 0.960 | 73.0 |
| 21 | 0.368 | 65.8 |
| 22 | | 10.1 |
| 23 | 1.777 | 70.4 |
| 24 | 3.75 | 80.7 |
| 25 | | 0 |
| 26 | | 0 |
| 27 | 0.372 | 68.7 |
| 28 | 0.059 | 61.9 |
| 29 | | 36.0 |
| 30 | | 0 |
| 31 | 2.472 | 79.8 |
| 32 | 0.917 | 63.5 |
| 33 | | 57.8 |
| 34 | | 58.5 |
| 35 | 2.491 | 77.1 |
| 36 | 2.179 | 77.3 |
| 37 | 0.947 | 70.0 |
| 38 | 3.192 | 80.8 |
| 39 | | 57.9 |
| 40 | 2.427 | 88.6 |
| 41 | | 59.0 |
| 42 | | 17.0 |
| 43 | 13.808 | 58.8 |
| 44 | 11.445 | 68 |
| 45 | 7.336 | 88 |
| 46 | | 24 |
| 47 | | −4 |
| 48 | 14.167 | 87 |

MIC Determination

The in vitro activity of compounds (at 10 μM concentration) against M. tuberculosis H37Rv was determined using the resazurin reduction microtiter assay (REMA) as previously described (Palomino, J. C., et al. (2002); Antimicrob. Agents Chemother. 46: 2720-2722.). MIC, defined as the minimum concentration of drug that inhibits more than 99% of growth of M. tuberculosis was determined in a 96-well plate format, with 10 μL of drug and 90 μL of bacterial suspension (OD_{600 nm}=0.001). Compounds were serially diluted two-fold from 100 to 0.16 μM and rifampicin control (1 mM to 1 nM) was included in every plate.

To prevent evaporation, plates were sealed. After 7 days incubation at 37° C., resazurin (0.025% w/v) was added and incubated for 20 hours at 37° C. before fluorescence reading. Bacterial growth was determined following resazurin reduction by fluorescence (excitation 570 nm, emission 590 nm).

Genotoxicity Assay

The genotoxicity of the compounds was evaluated by the SOS chromotest on LB agar plate. (Quillardet, P., O. et al. (1982). SOS chromotest, a direct assay of induction of an SOS function in Escherichia coli K-12 to measure genotoxicity. Proc. Natl. Acad. Sci. USA 79:5971-5.) This colorimetric assay is based on the induction of the SOS function SfiA by DNA damaging agents, whose level of expression is monitored by means of a sfiA::lacZ operon fusion in E. coli PQ37. Briefly, compounds (10 μL of a 10 mM solution) were spotted on a LB-agar plate containing ampicillin 50 μg/ml, 0.006% bromo-chloro-indolyl-galactopyranoside (X-Gal) and E. coli PQ37 at OD_{600 nm}=0.04. Isoniazid and 4-nitroquinoline N-oxide were used as negative and positive controls, respectively. Genotoxicity of compounds was evaluated calorimetrically.

TABLE 5
The MIC values and genotoxicity data of the selected compounds.

| No | H37Rv inh % at 20 uM | H37Rv MIC90 | HepG2 TD50 |
|---|---|---|---|
| 1 | 100.6 | 2.765 | |
| 2 | 101.6 | 9.71 | |
| 3 | −7.2 | | |
| 4 | 39 | | |
| 5 | 99.2 | 16 | |
| 6 | 94.1 | 19.6 | 19.6 |
| 7 | 58.2 | | |
| 8 | −48.4 | | |
| 9 | 100.9 | 19.3 | 19.6 |
| 10 | −21.2 | | |
| 11 | −21.5 | | |
| 12 | 102.1 | 1.13 | 19.6 |
| 13 | −23 | | |
| 15 | 101.9 | 0.292 | 0.0458 |
| 16 | 100.5 | 11.75 | 2.37 |
| 17 | −1.4 | | |
| 18 | −8.6 | | |
| 19 | 100.5 | 19.45 | |
| 20 | 102.8 | 4.84 | |
| 21 | −3.7 | | |
| 22 | 102.5 | 7.24 | 9.81915 |
| 23 | 102.2 | 10.25 | |
| 24 | −20.4 | | |
| 25 | −9.8 | | |
| 26 | −34.2 | | |
| 27 | −11.9 | | |
| 28 | 0.6 | | |
| 29 | −14.2 | | |
| 32 | 100 | 18.5 | |
| 33 | −30.6 | | |
| 34 | −4.3 | | |
| 35 | −8.1 | | |
| 36 | −9.8 | | |
| 37 | 100.8 | 13.65 | 16.1 |
| 38 | 96.4 | 19.6 | |
| 39 | −22.2 | | |
| 40 | −8.5 | | |
| 41 | −4.8 | | |
| 42 | 24.2 | | |
| 43 | −9.2 | | |

1. A compound of general formula (I) and pharmaceutically acceptable salts, solvates, hydrates, regioisomeric and polymorphic forms thereof: 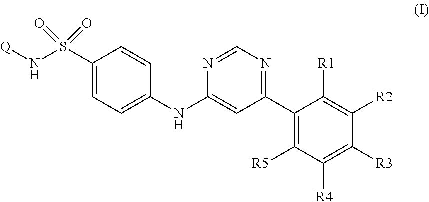
wherein
Q is a substituted or unsubstituted heterocyclyl having 5 or 12 ring member atoms where 1 to 3 of the ring member atoms are selected from the group of N, S and O and the other ring members are C, or alkanoyl, optionally substituted with one or more group selected from alkyl and oxo;
R1 to R5 are independently selected from the group of
a) hydrogen;
b) halogen;
c) optionally substituted alkyl, wherein the substituent is selected from the group of
optionally substituted aryloxy, wherein the substituent can be optionally halogen substituted alkyl or optionally halogen substituted alkoxy;
halogen,
aryl-alkoxy which is optionally substituted in the aryl part with optionally halogen substituted C₁₋₆ alkyl or optionally halogen substituted alkoxy;
d) optionally substituted alkoxy, wherein the substituent is selected from the group of
halogen,
optionally substituted aryl, wherein the substituent can be optionally halogen substituted alkyl or optionally halogen substituted alkoxy);
e) optionally substituted aryl;
e) aryloxy;
f) nitrile;
g) amine, which optionally substituted with 1 or 2 alkyl or alkylcarbonyl;
h) carboxamide;
i) or any 2 adjacent groups of R1 to R5 form together an alkylenedioxy;
k) or any 2 adjacent groups of R1 to R5, together with the atom to which they are attached, form a condensed benzene ring.

2. A compound according to claim 1, wherein Q is selected from the following group:
3,4-dimethyl-isoxazol-5-yl,
5-methyl-[1,3,4]thiadiazol-2-yl,
5-methyl-isoxazol-3-yl,
3,4-dimethyl-isoxazol-5-yl.

3. A compound according to claim 1, wherein in the meaning of R1 to R5, in point c) the aryl-alkoxy is a benzyloxyalkyl group.

4. A compound according to claim 1, wherein in the meaning of R1 to R5, in point d) the alkoxy optionally substituted with aryl is a benzyloxy group.

5. A compound according to claim 1, wherein in the meaning of R1 to R5, in point e) the aryloxy is a phenoxy group.

6. A compound according to claim 1 for use in the prevention and/or the treatment of cancer diseases.

7. A compound according to claim 1 for use in the prevention and/or the treatment of bacterial diseases, e.g. mycobacterial diseases.

8. A compound according for use according to claim 7 where the bacterial disease is tuberculosis.

9. Pharmaceutical composition containing as active ingredient one or more compound(s) of general formula (I) according to claim 1 together with one or more usual pharmaceutical auxiliary material(s).

10. Method for the prevention and/or the treatment of a cancerous disease where a compound of general formula (I) according to claim 1 is administered to an individual in need thereof.

11. Method for the prevention and/or the treatment of a bacterial diseases, especially mycobacterial diseases, e.g. tuberculosis, where a compound of general formula (I) according to claim 1 is administered to an individual in need thereof.
